SEQUENCE LISTING (30 sequences)

SEQ ID NO: 1 - DNA, 1683 bp, Pseudogymnoascus destructans
  source 1..1683: mol_type = genomic DNA
atgataatctctgaagtgattccctgctgccgtctccttccgccgttccaccacataaagtacataaacaccctcgtccagctgcgcagcttcaacagcccgcccaccaccgcccaagtcagcgtcccagccaaaattgcgtcgcctgtctcgcgccccttcacctccctcctcgctcccacaatcgacacccagtacccctccatgacggcgacgcagaccacgcacgagtcactctcgcagcggcgccgaatgcaccacgaagccgatgttgttataatcggggcgggcgtcttcgggtgcgccgtggcggtcacgttggcgcgccagggccgcagcgtcattctcctcgagcggtcgatgaaggagcctgaccggatcgtgggggagctgctgcaacccggcggcgtgtcggcattacacaagctgggattgtcggagtgtctggagggcattgatgcggtggttgttaatggctataatgtcatctactacgggaaggaggttcatatcccgtacccgtacgatacgagggtggagaagtcgagggcgatgagggagtcaaggccggtggggtggtgttttcaccacggacggttcattaacaagctccgcgaggcgtgcaagagcgagccgaatatcacgatcttcgagacgacggtgaaggggacggtgagcacggataacaacgagcaagtcctcggcgtaaagacagagacgacagaccccatggacgggtcgaagaaaccggattacttcttcggcggcctcaccatcgcagcagacggctacgcatccaccctgcgaaaacagtacatctccaagacccccgtggcgaagtccaaattctacgccctcgaactcatagactgccccatcccaaccccgaatcacggccacatcatcctctccgacaatgcccccgtcctcgtctaccagatcggcacccacgagacccgcgcgctcatcgacgtccccgagaacctggagaccgccaaagccgccctggggggcgtaaaagcacatattcgcaatgtcgtcgtgccaacgcttccgaagcaaatcaagccgacttttataagggcgctggaggaggggaaactaaggagtatgccgaattcgtggttgccgccgacgcagcagcagaccccggggattgtggtactgggggatgcgatgaacatgcgtcaccccctcaccggcggcggcatgtccatcgccctcaccgacgtggtcatcctctccgagctcctccacccgactcgcatcgcggacctctcctccgtccccgccatgagcctcgcaatgcgcaccttccactggcggaggaaacaactcgcatccatcgtgaacatcctcgcccaagccctctacgccctcttcgccgccaacaacgccgagctgaaagccctgcagcgggggtgctttgagtacttcctctttgggggcgcgtgcattgatgagccggcggggatgctggcatgtatcctgccgaggccgtttttgctgttctaccactttttctcggtggcgctgctagcgatgtggttgattatgtgtgattgtgtgggggatttgctgggggtgtggagggcgccgctgggggtgtataggagtggtgcggtgttgtggaaggcggtgggggtgatttttccgtatattttttccgagttgacgtggtga

SEQ ID NO: 2 - DNA, 258 bp, Pseudogymnoascus destructans
  source 1..258: mol_type = other DNA
ctgctgccgtctccttccgccgttccaccacataaagtacataaacaccctcgtccagctgcgcagcttcaacagcccgcccaccaccgcccaagtcagcgtcccagccaaaattgcgtcgcctgtctcgcgccccttcacctccctcctcgctcccacaatcgacacccagtacccctccatgacggcgacgcagaccacgcacgagtcactctcgcagcggcgccgaatgcaccacgaagccgatgttgttataat

SEQ ID NO: 3 - DNA, 1566 bp, Pseudogymnoascus destructans
  source 1..1566: mol_type = genomic DNA
atgactttcggtactacctcacagaatctctacaacgctcaaagacgctccgagcaccatgaggccgatgtcctggtggtcggcgctggagtgtttggctgcgcgatggcttatgcgttagctaaccaagggaggagtgttattctgctggagcggcagatgaaagagccggaccgcattgtcggggagcttctccaacccggtggtgtggaggcgttgaagaagcttgggctagagaaatgcctggaaggtattgatgccattccttgctacggctacaacgtccactaccacgacgacgacgtcgtgatcccgtaccctgccatcgagccttcgggtaaggtcttagcggccactcacagaggccttactgcccaagagtacttgcaggcggttggtaggaacaatacaaatggccatgcagataaagctagtggagccgtgcacaagttctctctcgacaagcccgagggccgtagttttcaccacggacggtttattatgcggctgcgacaatcctgcgacaatcacccaaacgtcactattttcgagacagaagtgacgaacacgatccggtgcgagcacacctctcagatcctcggtgtggagacgaagagagcggacaaaacaaaggattacttttttggccagctcactgtcatcgccgacgggtacaattccaggttccgcaagcagatcatcaacaagactcccattgttaagagcaagttttacgccttgacgcttatcgactgccccatgaagcctacgaattttggccacgtcatcattggcaaagctttccccattctaatgtaccagattggcaaccacgagacgcgcgcgctaatcgacgtgcccgagaacctcccggaggcatcccctcagaacggtggcgttcgtggctatatcagaaacgtcgtgattccaactcttccagaaggaacaaagcgggcatttgagttggccctggctagtggccgtatcccgaagagcatgccaaacagctggctcccaccggtgcgccagaagaacaacttgggcgtcgtcctcctcggcgacgcttacaatatgcgtcacccactaacaggcggcggcatgactgtagccttcaatgatgtcgtcctcctctcgaaattactccatccggacaaggtcgctgacctcggcaacaccgacgccattaacgcggccatgagcaagttccactggcagcgcaagtcgctcaccagtattattaacgtgcttgccatggcgctttatatgctctttgcagcacaggaccgccagcttgctgctttgcagcgcggctgcttcgtctacttcaaaaagggcgttactgatgagcccagcgccatgctaggcggtatcctccaccggccctctgtgctggcataccacttctttgccgtagctttcctatctatcaggatcaacaccgtcgacctttgcggggggggcatctccggcatttgcaaggtaccccgagccgttctcgacgctgtgctgatcctctggaaggcgtctgttgtctttctgcccgtcatgtggcaggaactgcagtaa

SEQ ID NO: 4 - DNA, 200 bp, Pseudogymnoascus destructans
  source 1..200: mol_type = other DNA
agctcactgtcatcgccgacgggtacaattccaggttccgcaagcagatcatcaacaagactcccattgttaagagcaagttttacgccttgacgcttatcgactgccccatgaagcctacgaattttggccacgtcatcattggcaaagctttccccattctaatgtaccagattggcaaccacgagacgcgcgcgcta

SEQ ID NO: 5 - DNA, 1590 bp, Pseudogymnoascus destructans
  source 1..1590: mol_type = genomic DNA
atgggcctaacagagagccttactgaggccgtcctgatcccaatcaatgcgcagatttcggagcgtggtctcggcgtcgtggcagccgtcggattcgcgtcgttcatcgtcctcgctgtcgtcctcaatgtcttgagccagcttcttccccaaaaccccaatgagccgccgcttgttttccactggttccccatcctcggaaacactatcagctatggaatggacccataccccttcttcttcaagtgcagagaaaagtatggtgatatctttacttttatcctcctcggaaagaagaccaccgtataccttggaacagctggcaacgagttcatcctcaacggcaagctcaaggacgtgaacgcggaggagatctacacaaagctcaccacgccagttttcggtaccggagttgtctacgactgccccaactcgaagctcatggagcagaagaaattcgtgaaagtcagtctcaccaccgagacattcaggtcctacgtgccccttatcgtccaggaggtcaccaacttcatcaagaccagcccgatgttcaagggtcccagcggtaaaaccgacgttcccgcagccatgtctgagatcaccatctacaccgcttcccgtacactccaagggaaggaagtccgatccaggttcaacgccgaatttgccaacctcttccacgacctcgacatgggcttcaccccgatcaacttcatgcttcactgggcacctctgccacaaaaccgtgctcgcgatcgcgctcagagaatcctttccgagacatacatggagatcatccaggaacgtcgcgccggaaacgtccctgatgagaatgagcacgatttgatccgacatctgatggcatcggtttacaaggacggcacccctctccctgacaaggagatttcacacatgatgattgccatgctcatggcgggacagcactcctcgtcatcgaccagctcctggatgatgcttaacctcgccgcccgccccgatataatcgagggactctaccaagaacagatccgcctcctaggcgccgacctcccccccttgacctatgagaacttgggcaagctcaccctgaacaacgccgtcctcaaggagaccctccgtctccacacccccatccactccatcctccgcaaagtcaagtcccccatgcccgtccccggaaccaacttcgtcatcccaacaacccacaccctcctctcctccgccggtgtcaccgcgcggatggaggagtacttccccgaccctctcaagtgggacccccatcgctgggacgcaggcgcagccggcgtccagatgggcgagatcaaggaggagcacgaggactacggctacggcatgatcagcaagggcgccacgtcgccctacctgccctttggcgctggccgccaccgctgcatcggcgagcagttcgccggcctgcagctcggggccatcatcgcgaccatggtgcgcgagttcaagtggaggctgcctgacggcgtgaacgaggttgtcggcacggattacagctcgctgttctcgcggccgctgaccccggccatgattgtgtgggagaagcgggagaagaactaa

SEQ ID NO: 6 - DNA, 219 bp, Pseudogymnoascus destructans
  source 1..219: mol_type = other DNA
atcgagggactctaccaagaacagatccgcctcctaggcgccgacctcccccccttgacctatgagaacttgggcaagctcaccctgaacaacgccgtcctcaaggagaccctccgtctccacacccccatccactccatcctccgcaaagtcaagtcccccatgcccgtccccggaaccaacttcgtcatcccaacaacccacaccctcctctcctcc

SEQ ID NO: 7 - DNA, 1464 bp, Pseudogymnoascus destructans
  source 1..1464: mol_type = genomic DNA
atggccggttcgaaatctaaagcccccgccgtgaaggcgcaacagaaacatggatatgagttcgctggaccgcccggtgcattcgccatctcgttcctccttccgatcgtcgtctacatcaccaatttcgtctgcaacgacatttacggatgcccgatcccctcggtgctggaccccaagacgttgacatgggacaagatcaagactgagactggatggccaggatggaatggcattgtgagcttcgaggctactggctgggtgttggcatactatttcctgagtttggtgctgcacagattccttccgggacaaatcgttgaaggaactgaactggctattggaggacgcttgaagtataagttcaatactctttcctcctccatctttaccgtcgttctcctcctcgccggtactatcgcgcagggcgccgacttcccagtctggacctacatctgggacaactacacccagatcgtcaccgccaacatgctcatcgccttcaccctcgcaacattcgtctacatccgcagcttcagcgtcaagcccggcaacaaagacatgcgcgagctcgccgccggtggccactccggcaacatgctctacgactggttcatcggccgcgaactcaacccgcgcgtcaccctccccatcttcggcgagatcgacatcaaagtcttctgcgagctgcgcccaggtctctttggctggatcctcctcgatgtcgccttcatcgcgcaccaatacaagacatacggccacgtcaccgactccatcatcctcgtcaccctcttccaggccctctacgtcttcgactccttctacatggaaccctccatgctgaccaccatggacatcaccaccgacggcttcgggttcatgctctccttcggtgacctcgcttgggtccccttcatctacagtctgcaagcgcgctacctcgccgtctaccccctcaccctcggcctctccggcaacgccggcgtcctcgccatcctcagtctggggtactacatattccggagcgccaacaaccaaaagaaccgcttccgcaccgatccgtctgacccgcgcatcgcgcatctcaagtacatggagacggcctcggggtcaaaactcatcatctccggatggtgggggacggcgcgccacatcaactaccttggcgactggatcatgtcgtgggcgttctgtctgcctaccgggatagcggggtaccttgtgcagcagggcccggcgctgttggatggcgagacgggattctcgcgcggggaggatagggtggtgcaggggggtgcgagggggtggggaatggtgatttcgtatttttacgttatttactttgcgattctgttgattcatcgcgagaggagagatgagacgaagtgtaagaggaagtatgggaaggattgggaggagtataagaagattgtgccgtggaggattattccagggatttattag

SEQ ID NO: 8 - DNA, 245 bp, Pseudogymnoascus destructans
  source 1..245: mol_type = other DNA
gttgaaggaactgaactggctattggaggacgcttgaagtataagttcaatactctttcctcctccatctttaccgtcgttctcctcctcgccggtactatcgcgcagggcgccgacttcccagtctggacctacatctgggacaactacacccagatcgtcaccgccaacatgctcatcgccttcaccctcgcaacattcgtctacatccgcagcttcagcgtcaagcccggcaacaaagacat

SEQ ID NO: 9 - DNA, 3360 bp, Pseudogymnoascus destructans
  source 1..3360: mol_type = genomic DNA
atgtatcgcccaaatacacctccaaacttcgatcctccatcatacggtgatgatgagggatcaacaccaacacacatgtcgaacgatgcgtccgccatccgcctgttgacatcaatggacgactccaactcctcgaacgcccgcatgtatgtacaggatcctgatttactaccccgtcctctcgcggtgcacaagaagtctgtgcagttctccacgctagaacacatttctgcccttcatggcgaagaagaagctatctcgagcggcaagttgccagctgcggtgaccaagcaagcgccgccgaagcccccccaagaagaccacttcgtgaatctcctccccaaactccctgatggaccctcgcgcaggaagtcgctgcggagtcaagtcaaagcccacttggctgctaacggtgagatgaagaatgaaagcttacctgtcaatagtcccagcaccccgcgcagatcataccagcctaccgtcatgtcgacgagctcgcgctccaattccatcgttgatggagcgccgatattaccaccggagagctcatacgacccatacaccgggagacagtcaccgacgcggtcgtggaccccatctcaaacagcgtctgagagaggtaggccaccatcgatagctcaatatgagccagcggatgtgaatggctctccgagacccggcactccttcgtcgagatacggcggcagcccgaggagaccactccctcccgcaccactattctctggaccgggtgctggtgcgcggagctccacattcgcagacgacgcgacggtttcgatcgcgttagaaacagaaggagacgacgtttttgctccgaaaagtactatcggtactgatcatgtccggtcccagtctcgcgagtcctacacgtcagagtcgacgttcaccgaggagtacgacaatgagaagtccgactttgagcactacgggcctgcgccagatgggcgacaggagcgcagaggtgctcgccaggcacagatggccaagaaggaggttcgtctcattaacggcgagcttatcctcgaatgcaagatccccaccatcttgtacagtttcctcccccgccgcgatgaggttgagttcacgcacatgagatacacagccgtgacctgcgatcccgatgacttcgtgtccaagggctacaagctgcgccagaacattggctcgacggccagggaaacagagctcttcatctgtatcaccatgtataacgaggacgagatcaacttcacgcgcaccatgcacgccgtcatgaagaatattgcccatttctgctcgcgctccaagtcgcgcacgtggggcgagaacgggtggcagaagattgtcgtctgcatcgtctctgatggtcggcagaagatccacccgcgcacgctcgatgcgctcgcggcgatgggtgtgtaccaggacggcattgccaagaatcttgtcaaccagaaggaggtgcaggcgcacgtgtacgagtataccactcaggtgtcgctggactcggacctgaagttcaagggcgcggagaagggaatcgtgccggtgcagatgctgttctgtctgaaggagaagaacgcgaagaagctgaactctcatcgctggttcttcaatgcttttggtcgcactctgacgccgaatatctgcatcctgcttgatgtgggtacgaagccgacggggaattctctttaccatctctggaaggcctttgacaccgactccaacgtcgcaggtgcctgtggagagatcgtggcgatgaaggggaagggctggcttgggttgttgaacccgctggttgcgtcgcagaattttgagtacaagatgtctaatatcttggataagccgctcgagtcggtgtttggatacattactgtccttcctggtgctctcagtgcgtatcgttaccacgctctgcagaatgaccatactggccatggaccgctaagtcagtacttcaagggtgaaactctgcacggccagaatgccgatgtcttcacggccaatatgtatcttgctgaagatcggattctgtgctgggagcttgtcgcgaaacgagatgagagatgggtgttgaaatatgtcaagaactgcaagggagagactgatgtgcctgataccgtccccgagtttgtctcccagcgtcgccgttggctcaatggagctttcttcgctgccgtctactctctggtgcatttcaagcagatctggaacacagaccacaccatcgcacgcaagatcctcctccatattgaattcgtgtaccagctgctcagtctgctcttcacgttcttctccctggcgaacttctacctcacgttctactttgttgctggctcgctcgctgatcccaaggtcgatccgtttggccacagcatcgggaaatacatcttctatatcctgaaatatgtctgcgtgcttctcatctgcacgcaattcatcctctccatgggcaaccggccacagggcgcgaagaagttgtacctttctagtatggtcatctacgcaatcatcatggtatacacgacctttgcgacgatctacatcgtcgtgcggcagtttaccagcaacaccataactctcggaaacaacatcttcaccaacgtcgccgtgtccatcgcctcgacgctcgggctgtacttcttctcctccttcctatacctcgacccatggcacatgttcacctctgcggcgcagtacttcgccctcctgcccagctacatctgcaccctccagatctacgccttctgcaacacccacgacatctcctggggaaccaagggcgacaacaccgttcgcaccgacctcggcacagccgtctccaaacacaagggctccaccgtcgagctcgacatgcccagcgagcagctcgacatcgactccggctacgacgaggccttgcgcaacctccgcgatcgcatcgaggttcccgagctcggcccatccgaggagaccgcacaggaggactactaccgcgctgtgcgcacgtacatggtcgtctcgtggatggtggcgaatgccatcctggccatggccgtgtcggaggcgtaccagggcaagaacatcgggagcaactcgtacctcaagttcctgctttggtctgtggcggcgatcgcgctgttccgcgcggtgggtagcacgacgttccgcatcattgagctggtgggtatgcttattgacgggaaggcgaagtgggagagcgggagctacaggtgggggggtagctcggtgggggggagcacggtgcttagctcgaaggctagggggggtgggttttggtcgaagtttgggtttgggagtgtcaaggataagattagtgatatctcgagtagtatcgggagcagtgtttcgaggaagtga

SEQ ID NO: 10 - DNA, 243 bp, Pseudogymnoascus destructans
  source 1..243: mol_type = other DNA
tctcatctgcacgcaattcatcctctccatgggcaaccggccacagggcgcgaagaagttgtacctttctagtatggtcatctacgcaatcatcatggtatacacgacctttgcgacgatctacatcgtcgtgcggcagtttaccagcaacaccataactctcggaaacaacatcttcaccaacgtcgccgtgtccatcgcctcgacgctcgggctgtacttcttctcctccttcctatacct

SEQ ID NO: 11 - DNA, 2718 bp, Pseudogymnoascus destructans
  source 1..2718: mol_type = genomic DNA
atggacggtcgttacaatccaggggcatctcttcgtgaggtggatccttcgaatcctggatataaccaacctaggccagacactcgcgatgaatccgaactgggcctgctatccccaagcggtgggcatgcataccagtccccatttgatggcaacaatcaatcgaacctggaaattcagaggcctgtctcgacagcatacagcttgacagagtcgtacgcggtgcctgggcagcagaacccacaccacgcaacagagtattcgagcagctcatccttccagcaaggaatcgaactcgaggacattcctttcggaggcacaaaccgagccccgtcgccatcgcgaacgatcgactcggaagatgcctggagaaagcgtcaggctcctggcggcggactcaagcgctaccctacaagaaaggtgaagcttattcagggctcggtcctcagtatcgactacccagtcccgagtgccatcaagaactcgatagagaagaaatacaccgccgatgtcgaagctgggaatgaagaattcacccacatgcgatacactgcggctacctgcgaccccaacgacttcacaatgaggaacggctacaatttgaggcccgcgatgtacaataggcacaccgagctcctgattgcaattacatactataacgaagataaggcgctgctttcgcgcacgctgcacggtgtcatgcagaacatcaacgacattgtccgtctcaagaagactgagttctggaacaagggaggacaggcatggcaaaagattgtcgtctgcttggtcttcgatggttttgatgcttgcgacaaggacgttcttgacgttctggccacggtcggtgtcttccaagaaggtgtcatgaagaaggatatcgacggtaaggagacagttgcccatatcttcgagtacacgacacagatctcggtgactgccaaccagcagcttgtgcgccctcaagagggtgcggcaaacaacctgccacccgtgcagatgatgttctgcttgaagcagaagaacagcaagaagatcaactcccatcgttggttgttcaacgctttcgggcgtattctgaacccagaggtctgtattctgctggatgccggtactaagcctggtcccaaatcgctcttgtcgctgtgggaaggtttcttcaacgacaaagatctcggaggagcttgtggtgaaatccacgccatgttgggcaagggtgggcgcaacctgatcaaccctctggttgcagcccagaacttcgagtacaagatcagtaacattctcgacaagccgcttgagagttctttcggatatgtcagtgtgttgccgggtgctttctcagcataccgttaccgtgctatcattggccgcccgctcgagcagtacttccatggtgatcacacgctttctgcgattcttggaaagaagggtatcgagggcatgaatattttcaagaagaacatgttcttggctgaggatcgtattctttgcttcgagttggttgccaaggctggctcgaagtggcatttgacatacatcaaggcggcaaagggagaaacggatgttcctgagggcgcccctgaattcattggccagcgtagaagatggctcaacggttcatttgctgccagtgtctactccgttatgcactttggtagaatgtacaagagtggacacaacttcgtccgcatgttcttcttccatatccaacttctttacaacatcttcacggttatcctcacttggttctcgctcgcatcttactggcttacaacgactgtcattatggatcttgttggtcttccagagccagcttcgggtaccggagcagagcatcacggatggcctttcggagacacagcgacgccaattatcaacacgattctgaagtacctatacctcggatgtctggtcatgcaattcgtgttggccctcggaaataggccaaagggatcccgctacacttacatcacatcgttcatgatcttctcattcattcagttgtatgtcattattttgtctatgttcttggttgtacgcgcgtttgtcggaccaaacgtggacaaaacagcgatcttcaccaccaatatcttctcgtcaaacagctccgctatcattctcatcgcgctggcggccaccttcggtttatactttgtcgcctcgttcctctacctggacccatggcacatgttccactccttccctcaatacctgcttgttgcatcttcgtacatcaacatcctcaacgtctactcctacagcaactggcacgatgtgtcttggggaaccaagggatcggacaaggtcgatgctttgccatctgcacagacaaccaaggttgagggcaaggctgctgtcatcgaagaagtcgacaagccacaagaggatattgatagccagtttgaggcaacagtcaagcgcgctctcaaggaatacaaggcacccaaggaggatgagtccaagtctttggaggattcgtacaagtctttcagaacgaccctggtggctttctggattttctccaacgccattcttgcggtggtcattactagcgacaacttcgattcgttcggattcgggaacaaagcatctgctcgcacagccaagttcttccaggccctgctcatcgctactgtctgtctgtctctcgttcgctttattggatgcctgtggttcctgggtaagtccggaatcaagcgctgcttcaggagacgttga

SEQ ID NO: 12 - DNA, 250 bp, Pseudogymnoascus destructans
  source 1..250: mol_type = other DNA
ttcgtgttggccctcggaaataggccaaagggatcccgctacacttacatcacatcgttcatgatcttctcattcattcagttgtatgtcattattttgtctatgttcttggttgtacgcgcgtttgtcggaccaaacgtggacaaaacagcgatcttcaccaccaatatcttctcgtcaaacagctccgctatcattctcatcgcgctggcggccaccttcggtttatactttgtcgcctcgttcctct

SEQ ID NO: 13 - DNA, 3627 bp, Pseudogymnoascus destructans
  source 1..3627: mol_type = genomic DNA
atgtccctaccccagagacctgggggcggcgacctccccccccaagaacggcgaaatacgtatagaagctccactgggcgccctcaaccgtcacacgacgccgaagttggatatgctgggcaagattcgcaatctccaacatctagacggacaagacaacatcggacagaacgagccgggtctcccgctgatactcgcccgcctcagtcccccacccccgcaactccagctgcacaggatttccaacgcaagcgaagcttgatccgccctgagagaaaccgcattgaccgcgaccatccaaactactattatcgccaacatgcggcgaatatgacagttttgccttcgactacgggaaacgacccgattctagaagatcaaggagatataaatggcgggagtggcgagccggatcagcgcagtcctcgggatggtccctatgggtctccatcgcctctggaatcccaaggaacgatgcaaggttcagacaccggcaacttcgaaaaggaccataaatcctcaaagctaactcgtgacacaaacaagtcgaggaagcagacaagagaagaaagacgccgccagagggacgcagaagtcctcaagccgcctagtttgtggaacgtgtattgctcgattatcacattctggtgccctgattttattctgaggtgcttcgggaagccagcgaaagaacaacagagagcatggagggagaagatgggacttatcagcattattctcatggtcatggccttcgtcggctttctcacgtttggattcactgccactgtgtgtggtaacccaccaacccgactccgcgtaaatgaggtcgacaaaggatatatgatcttccacggcaaagcgtataatttattgggggttcgtcatcctgcggccatgggtattccggaacagtcaaatatcttgtatgtgcctgaaagtcatggaggcatggacggcagttttcttttccaaaacgtcaacggaaagtgcaaaggcttgatctcaaagtcagagggatccgacgttccaagtgatgaccagggaaacctcggttggtacttcccatgcaaccttctgaaccaggacggctccagtgatcctaaaaagaagagcttcggaccctaccttggccatcaatgccataccaccgcacctggacgaactgcattctatggcatgcactctgcgggggatgtctactacacctgggatgatatcaacaatggtacaaggaatttggctgtctactctgggaatgtgttggacttggatcttctgaaatggtttaatgggtcgcaggttactgtccctcgaatttttgtcgacctcggcaacaaggctaccaccctgaaccaaggaatcagaggtagagacataacacataaattccaagcctctggagacaaggaacttgccgaatgtttagaacagatcatcaaggttggaagcgtcgataccgaaacgattggctgtatcgcgtcccaagtggtgctttacgtagcgctcgcattcatcatctctattgttggagcaaaattcgtcctcgctctctacttccagtggttcttgagccgcaggtatgcggccagtaagacctcacaatcttcggatccaaagaaacgtaacaggcagatcgaggagtggacaaacgacatctatcgcgcccctcctcgcattgcgggagaccccggcagcactgctgctggttcctcggacagaaatagcagacgcgccagcagcatgttccttccaaccacgtctcgcttcacaagcccgtatacactaaatggagagaagtcaggaccacggccggcaccgacgactatggcgagccagaactcagcagctcagctattcccgccaaacccgatgtatagaggccaaaacgatagccgaatgagcttccctaattcgaacctagatggcggtgttatgagcgatgtatcaggcgacgggccaggccctgctggcttcatccatgaagctgtcgtgcctcagcctcctccagagtggcaaccatttggatatcctcttgcacacgcgatctgccttgtcaccgcttactcagaaggtgagcagggtattagaaccacactcgactctgtagcgacaaccgattaccccaacagccacaagatgatactcgttatctgtgatggtatgatcaaaggaaagggcgagctacattctacccctgaaattgtcttgggcatgatgaaggatcactctgtgctgcctgaggacgctccggccttcagctacgtcgccgttgcaagtggatcgaaacgacacaacatggcaaaggtatattctggattctacgattatggcgccgactcggcaattcctttggatcgccaacagagagtgcccatgatgtgtcttgttaagtgcggaacccccgatgaagcatcgaagagcaagcctggaaacagaggaaagcgtgactcgcaaattatcctcatgtctttcctccaaaaggttatgttcgacgagcggatgacggagctcgagttcgagatgttcaacggcttatggaaaatcactggaatgtcaccagacttcttcgaggttgtgttgatggttgatgccgataccaaggtcttccccgacagcttaactcacatggtttccgccatggtcaaggacccagagatcatgggattgtgtggtgagacgaagattgcaaacaagagagtctcctgggttacagccatccaagtcttcgaatacttcatctcccatcatctctccaaatctttcgagtccgtctttggtggcgttacttgtctccctggatgtttcagtgtgtaccgaatcaaggcacccaagggtggacaaaactactgggtccctatcctcgccaaccctgacgtcgttgagcactactctgagaacgtggtcgatacactacacaagaagaacttgttgttgcttggtgaagatcgttacctctcaacactcatgctcaggactttcccgaagcgcaagcaggtctttgtgccacaggccgtatgcaaaactactgtgcctgagcaattctcggtattgttgtctcagagacgtagatggattaacagtaccatccacaacttgatggaactggttctcgtcagagatctatgtggtactttctgcttcagtatgcagttcgtggtgttcatcgatttgatcggaacacttgtcctgcccgctgctattgcgttcactatctacgtcgtcgtcatatccatcgtcagaaagcccgttcaagtcatccccctcgtcctcctcggcctaatcctcggtctcccagccgtcctgatcgtcctcaccgcccacagatggtcctacgtcgtctggatgttaatctacctgatctccctccccatctggaacttcgtactcccggtctacgcctactggaagttcgacgacttctcgtggggcgacacgcgcaagacggccggcgagaagaccaagaaggccggcatcgagtacgagggcgagttcgacagcagcaagatcaacatgaagcgctggggcgagtttgagaaggagaggagacagagacaggccgggcagtggaatgcgcatagcaacatcagcagctatagggacgactactatgataataataattaa

SEQ ID NO: 14 - DNA, 290 bp, Pseudogymnoascus destructans
  source 1..290: mol_type = other DNA
gggcggcgacctccccccccaagaacggcgaaatacgtatagaagctccactgggcgccctcaaccgtcacacgacgccgaagttggatatgctgggcaagattcgcaatctccaacatctagacggacaagacaacatcggacagaacgagccgggtctcccgctgatactcgcccgcctcagtcccccacccccgcaactccagctgcacaggatttccaacgcaagcgaagcttgatccgccctgagagaaaccgcattgaccgcgaccatccaaactactattatc

SEQ ID NO: 15 - DNA, 1812 bp, Pseudogymnoascus destructans
  source 1..1812: mol_type = genomic DNA
atgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccgggcagttttaacgatcagttcgccgatgcagatattcgtaattatgtgggcaacgtctggtatcagcgcgaagtctttataccgaaaggttgggcgggccagcgtattgtactgcgtttcgatgcggtcactcattacggcaaagtgtgggtaaataatcaggaagtgatggagcatcagggcggctatacgccatttgaagccgatgtcactccgtatgttattgccgggaaaagtgtacgtatcactgtttgtgtgaacaacgaactgaactggcagactatcccgccgggaatggtgattaccgacgaaaacggcaagaaaaagcagtcttactttcatgatttctttaactacgccgggatccatcgcagcgtaatgctctacaccacgccgaatacctgggtggacgatatcaccgtggtgacgcatgtcgcgcaagactgtaaccacgcatctgttgactggcaggtgggggcaaatggtgatgtcagcgttgaactgcgtgatgcggatcaacaggtggttgcaactggacaaggcaccagcgggactttgcaagtagtgaatccgcacctctggcaaccgggtgaaggttatctctatgaactgtgcgtcacagccaaaagccagacagagtgtgatatctacctgctgcgcgtcggtatccggtcagtggcagtgaagggcgaacagttcctgatcaaccacaaaccgttctactttactggctttggtcgtcatgaagatgcggatttgcgcggcaaaggattcgataacgtgctgatggtgcacgatcacgcactaatggactggattggggccaactcctaccgtacctcgcattacccttacgctgaagagatgctcgactgggcagatgaacatggcatcgtggtgattgatgaaactgcagctgtcggctttaacctctctttaggcattggtttcgaagcgggcaacaagccgaaagaactgtacagcgaagaggcagtcaacggggaaacccagcaggcgcacttacaggcgattaaagagctgattgcgcgtgacaaaaaccacccaagcgtggtgatgtggagtattgccaacgaaccggatacccgtccgcaaggtgcacgggaatattttgcgccactggcggaagcaacgcgtaaactcgacccgacgcgtccgatcacctgtgtcaatgtaatgttctgcgacgctcacaccgataccatcagcgatctctttgatgtgctgtgcctgaaccgttattacggttggtatgtccaaagcggcgatttggaaacggcagagaaggtactggaaaaagaacttctggcctggcaggagaaactgcatcagccgattatcatcaccgaatacggcgtggatacgttagccggcctgcactcaatgtacaccgacatgtggagtgaagagtatcagtgcgcctggctggatatgtatcaccgcgtctttgatcgcgtcagcgctgtcgtcggtgagcaggtatggaatttcgccgattttgcgacctcgcaaggcatattgcgcgttggcggtaacaagaaagggatcttcactcgcgaccgcaaaccgaagtctgcggcttttctgctgcaaaaacgctggaccggcatgaacttcggtgaaaaaccgcagcagggaggcaaacaatga

SEQ ID NO: 16 - DNA, 237 bp, Pseudogymnoascus destructans
  source 1..237: mol_type = other DNA
caactcctaccgtacctcgcattacccttacgctgaagagatgctcgactgggcagatgaacatggcatcgtggtgattgatgaaactgcagctgtcggctttaacctctctttaggcattggtttcgaagcgggcaacaagccgaaagaactgtacagcgaagaggcagtcaacggggaaacccagcaggcgcacttacaggcgattaaagagctgattgcgcgtgacaaaaacca

SEQ ID NO: 17 - RNA, 258 nt, synthetic construct
  source 1..258: mol_type = other RNA
ctgctgccgtctccttccgccgttccaccacataaagtacataaacaccctcgtccagctgcgcagcttcaacagcccgcccaccaccgcccaagtcagcgtcccagccaaaattgcgtcgcctgtctcgcgccccttcacctccctcctcgctcccacaatcgacacccagtacccctccatgacggcgacgcagaccacgcacgagtcactctcgcagcggcgccgaatgcaccacgaagccgatgttgttataat

SEQ ID NO: 18 - RNA, 258 nt, synthetic construct
  source 1..258: mol_type = other RNA
attataacaacatcggcttcgtggtgcattcggcgccgctgcgagagtgactcgtgcgtggtctgcgtcgccgtcatggaggggtactgggtgtcgattgtgggagcgaggagggaggtgaaggggcgcgagacaggcgacgcaattttggctgggacgctgacttgggcggtggtgggcgggctgttgaagctgcgcagctggacgagggtgtttatgtactttatgtggtggaacggcggaaggagacggcagcag

SEQ ID NO: 19 - RNA, 200 nt, synthetic construct
  source 1..200: mol_type = other RNA
agctcactgtcatcgccgacgggtacaattccaggttccgcaagcagatcatcaacaagactcccattgttaagagcaagttttacgccttgacgcttatcgactgccccatgaagcctacgaattttggccacgtcatcattggcaaagctttccccattctaatgtaccagattggcaaccacgagacgcgcgcgcta

SEQ ID NO: 20 - RNA, 200 nt, synthetic construct
  source 1..200: mol_type = other RNA
tagcgcgcgcgtctcgtggttgccaatctggtacattagaatggggaaagctttgccaatgatgacgtggccaaaattcgtaggcttcatggggcagtcgataagcgtcaaggcgtaaaacttgctcttaacaatgggagtcttgttgatgatctgcttgcggaacctggaattgtacccgtcggcgatgacagtgagct

SEQ ID NO: 21 - RNA, 219 nt, synthetic construct
  source 1..219: mol_type = other RNA
atcgagggactctaccaagaacagatccgcctcctaggcgccgacctcccccccttgacctatgagaacttgggcaagctcaccctgaacaacgccgtcctcaaggagaccctccgtctccacacccccatccactccatcctccgcaaagtcaagtcccccatgcccgtccccggaaccaacttcgtcatcccaacaacccacaccctcctctcctcc

SEQ ID NO: 22 - RNA, 219 nt, synthetic construct
  source 1..219: mol_type = other RNA
ggaggagaggagggtgtgggttgttgggatgacgaagttggttccggggacgggcatgggggacttgactttgcggaggatggagtggatgggggtgtggagacggagggtctccttgaggacggcgttgttcagggtgagcttgcccaagttctcataggtcaagggggggaggtcggcgcctaggaggcggatctgttcttggtagagtccctcgat

SEQ ID NO: 23 - RNA, 245 nt, synthetic construct
  source 1..245: mol_type = other RNA
gttgaaggaactgaactggctattggaggacgcttgaagtataagttcaatactctttcctcctccatctttaccgtcgttctcctcctcgccggtactatcgcgcagggcgccgacttcccagtctggacctacatctgggacaactacacccagatcgtcaccgccaacatgctcatcgccttcaccctcgcaacattcgtctacatccgcagcttcagcgtcaagcccggcaacaaagacat

SEQ ID NO: 24 - RNA, 245 nt, synthetic construct
  source 1..245: mol_type = other RNA
atgtctttgttgccgggcttgacgctgaagctgcggatgtagacgaatgttgcgagggtgaaggcgatgagcatgttggcggtgacgatctgggtgtagttgtcccagatgtaggtccagactgggaagtcggcgccctgcgcgatagtaccggcgaggaggagaacgacggtaaagatggaggaggaaagagtattgaacttatacttcaagcgtcctccaatagccagttcagttccttcaac

SEQ ID NO: 25 - RNA, 243 nt, synthetic construct
  source 1..243: mol_type = other RNA
tctcatctgcacgcaattcatcctctccatgggcaaccggccacagggcgcgaagaagttgtacctttctagtatggtcatctacgcaatcatcatggtatacacgacctttgcgacgatctacatcgtcgtgcggcagtttaccagcaacaccataactctcggaaacaacatcttcaccaacgtcgccgtgtccatcgcctcgacgctcgggctgtacttcttctcctccttcctatacct

SEQ ID NO: 26 - RNA, 243 nt, synthetic construct
  source 1..243: mol_type = other RNA
aggtataggaaggaggagaagaagtacagcccgagcgtcgaggcgatggacacggcgacgttggtgaagatgttgtttccgagagttatggtgttgctggtaaactgccgcacgacgatgtagatcgtcgcaaaggtcgtgtataccatgatgattgcgtagatgaccatactagaaaggtacaacttcttcgcgccctgtggccggttgcccatggagaggatgaattgcgtgcagatgaga

SEQ ID NO: 27 - RNA, 250 nt, synthetic construct
  source 1..250: mol_type = other RNA
ttcgtgttggccctcggaaataggccaaagggatcccgctacacttacatcacatcgttcatgatcttctcattcattcagttgtatgtcattattttgtctatgttcttggttgtacgcgcgtttgtcggaccaaacgtggacaaaacagcgatcttcaccaccaatatcttctcgtcaaacagctccgctatcattctcatcgcgctggcggccaccttcggtttatactttgtcgcctcgttcctct

SEQ ID NO: 28 - RNA, 250 nt, synthetic construct
  source 1..250: mol_type = other RNA
agaggaacgaggcgacaaagtataaaccgaaggtggccgccagcgcgatgagaatgatagcggagctgtttgacgagaagatattggtggtgaagatcgctgttttgtccacgtttggtccgacaaacgcgcgtacaaccaagaacatagacaaaataatgacatacaactgaatgaatgagaagatcatgaacgatgtgatgtaagtgtagcgggatccctttggcctatttccgagggccaacacgaa

SEQ ID NO: 29 - RNA, 290 nt, synthetic construct
  source 1..290: mol_type = other RNA
gggcggcgacctccccccccaagaacggcgaaatacgtatagaagctccactgggcgccctcaaccgtcacacgacgccgaagttggatatgctgggcaagattcgcaatctccaacatctagacggacaagacaacatcggacagaacgagccgggtctcccgctgatactcgcccgcctcagtcccccacccccgcaactccagctgcacaggatttccaacgcaagcgaagcttgatccgccctgagagaaaccgcattgaccgcgaccatccaaactactattatc

SEQ ID NO: 30 - RNA, 290 nt, synthetic construct
  source 1..290: mol_type = other RNA
gataatagtagtttggatggtcgcggtcaatgcggtttctctcagggcggatcaagcttcgcttgcgttggaaatcctgtgcagctggagttgcgggggtgggggactgaggcgggcgagtatcagcgggagacccggctcgttctgtccgatgttgtcttgtccgtctagatgttggagattgcgaatcttgcccagcatatccaacttcggcgtcgtgtgacggttgagggcgcccagtggagcttctatacgtatttcgccgttcttggggggggaggtcgccgccc

(19) United States
(12) Patent Application Publication
(10) Pub. No.: US 2025/0368994 A1
(43) Pub. Date: Dec. 4, 2025

(54) COMPOSITIONS AND METHODS FOR CONTROL OF PSEUDOGYMNOASCUS DESTRUCTANS

(71) Applicant: Oregon State University, Corvallis, OR (US)

(72) Inventors: Jenny Urbina, Corvallis, OR (US); Emily Dziedzic, Corvallis, OR (US); Taal Levi, Corvallis, OR (US)

(73) Assignee: Oregon State University, Corvallis, OR (US)

(21) Appl. No.: 19/227,370

(22) Filed: Jun. 3, 2025

Related U.S. Application Data
(60) Provisional application No. 63/655,909, filed on Jun. 4, 2024.

(51) Int. Cl. C12N 15/113

(52) U.S. Cl. CPC C12N 15/113; C12N 2310/14; C12N 2310/315; C12N 2310/321

(57) ABSTRACT

Disclosed are compositions and methods for control of Pseudogymnoascus destructans (Pd) in the environment and infected hosts using RNA interference technology. In particular, trigger polynucleotides targeting fungal sequences and their use in prevention and treatment of Pseudogymnoascus destructans (Pd) infections in organisms are disclosed.

### CROSS-REFERENCE TO RELATED APPLICATION

This application claims the benefit of U.S. Patent Application No. 63/655,909, filed Jun. 4, 2024, expressly incorporated herein by reference in its entirety.

### STATEMENT REGARDING SEQUENCE LISTING

The Sequence Listing XML associated with this application is provided in XML format and is hereby incorporated by reference into the specification. The name of the XML file containing the sequence listing is 3014-P42US-Sequence-Listing.xml. The XML file is 50,282 bytes; was created on May 23, 2025; and is being submitted electronically via Patent Center with the filing of the specification.

### BACKGROUND

North American insectivorous bats consume large volumes of insects and are crucial for pest control in agricultural systems. An invasive pathogenic fungus, Pseudogymnoascus destructans (Pd), has been spreading across the continent for almost 20 years, devastating bat populations and several bat species are now threatened with extinction as a result (Frick et al. 2016). Bats become infected with the fungus when they are exposed to Pd, either via other bats or from hibernacula substrates. The fungus then infiltrates bat tissues below the epidermis, causing irritation and repeated emergence from hibernation. This depletes fat stores in bats, ultimately causing starvation and death. Numerous Pd controls have been proposed and studied and can be either indirect or direct. Indirect methods rely on introducing competitors such as the probiotic bacteria Pseudomonas fluorescens that compete with Pd to increase overwinter survival of bats (Hoyt et al. 2019). Similar indirect approaches have been proposed that rely on engineering a less virulent strain of Pd to compete with existing Pd (Flieger et al. 2016), but this would require the release of a transgenic organism and with it the risk that lower virulence Pd in higher abundances could produce similar devastating fitness consequences in bats. Methods to directly reduce the fitness of the fungus, such as treatment with ultraviolet light or fungicides, have been proposed (Chaturvedi et al. 2011; Palmer et al. 2018) but their efficacy in the field is not yet known (Palmer et al. 2018). In addition, many direct approaches kill or harm non-target organisms, which makes them inappropriate for Pd management in many sites (i.e., natural caves).

Despite the advances for Pd management, a need exists for improved targeted, direct control to reduce the fitness of Pd on substrates and within bat cells with minimal or no non-target effects. The present invention seeks to fulfill this need and provides further related advantages.

### SUMMARY

In one aspect, the disclosure provides a double-stranded ribonucleic acid (dsRNA) for inhibiting expression of a target gene in Pseudogymnoascus destructans (Pd). In certain embodiments, the target gene is selected from the group consisting of a gene involved in the synthesis of ergosterol and a gene involved in the synthesis of chitin, and wherein the dsRNA comprises a first strand comprising a region of complementarity that is substantially complementary to a target region of the mRNA encoded by the target gene.

In certain embodiments, the region of complementarity is from 85% to 100% complementary with the mRNA target region.

In certain of the above embodiments, the target gene is involved in the synthesis of ergosterol. Representative target genes involved in the synthesis of ergosterol include ERG1_1, ERG1_2, ERG_11, and ERG_24. In certain embodiments, the mRNA target region is encoded by a target gene sequence comprising or contained within the sequence shown in SEQ ID NO:2, SEQ ID NO:4, SEQ ID NO:6, or SEQ ID NO:8. In certain of these embodiments, the RNA sequence of the first strand comprises a sequence having from 95% to 100% identity to the sequence shown in SEQ ID NO:18, SEQ ID NO:20, SEQ ID NO:22, or SEQ ID NO:24.

In other embodiments, the target gene is involved in the synthesis of chitin. Representative target genes involved in the synthesis of chitin include CHS2_1, CHS2_2, and CHS3. In certain embodiments, the mRNA target region is encoded by a target gene sequence contained within the sequence shown in SEQ ID NO:10, SEQ ID NO:12, or SEQ ID NO:14. In certain of these embodiments, the RNA sequence of the first strand comprises a sequence having from 95% to 100% identity to the sequence shown in SEQ ID NO:26, SEQ ID NO:28, or SEQ ID NO:30.

In certain of the above embodiments, the dsRNA comprises at least one modified nucleotide. Suitable modified nucleotides include a 2′-O-methyl modified nucleotide and a nucleotide comprising a 5′-phosphorothioate group. In certain of these embodiments, the modified nucleotide is a 2′-deoxy-2′-fluoro modified nucleotide, a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, or a non-natural-base-comprising nucleotide.

In another aspect, the disclosure provides a composition for controlling Pseudogymnoascus destructans (Pd), the composition comprising a dsRNA as described herein.

In certain embodiments, controlling Pseudogymnoascus destructans (Pd) comprises inhibiting expression of a target gene in Pseudogymnoascus destructans (Pd).

In certain embodiments, the dsRNA is bound to a carrier to improve stability and/or delivery of the dsRNA. In certain of these embodiments, the carrier is a poly (butylene terephthalate) (PBT) nanocomposite, chitosan, a carbon dot, a silica nanoparticle, montmorillonite, kaolinite, a chitosan nanoparticle, a layered double hydroxide (LDH) nanoparticle, a liposome, a halloysite nanotube, lipofectamine, a nanoclay, an inorganic nanoparticle, a peptide, or a polymer.

In certain embodiments, the composition is formulated for administration by spraying or fogging.

In certain embodiments, the composition comprises two or more dsRNAs for inhibiting expression of two or more Pseudogymnoascus destructans (Pd) target genes.

In a further aspect, the disclosure provides a method for controlling Pseudogymnoascus destructans (Pd) in the environment, the method comprising administering to a substrate comprising Pd, or to a substrate susceptible to establishment of Pd, an effective amount of a composition for controlling Pseudogymnoascus destructans (Pd) as described herein.

In certain embodiments, controlling Pseudogymnoascus destructans (Pd) in the environment comprises inhibiting expression of a target gene in Pseudogymnoascus destructans (Pd).

### DESCRIPTION OF THE DRAWINGS

The patent or application file contains at least one drawing/photograph executed in color. Copies of this patent or patent application publication with color drawing(s) will be provided by the Office upon request and payment of the necessary fee.

The foregoing aspects and many of the attendant advantages of this invention will become more readily appreciated as the same become better understood by reference to the following detailed description, when taken in conjunction with the accompanying drawings.

FIG. 1 is a flow chart illustrating RNAi target development.

FIG. 2 is a fluorescent confocal microscope image visualizing labeled dsRNA in Pd cells.

FIG. 3 is a schematic representation of the ergosterol biosynthesis pathway (see Bhattacharya et al. 2018).

FIG. 4 illustrates the experimental design of pre- and post-germination experiments evaluating the effects of silencing genes in the ergosterol pathway (ERG_1_1, ERG_1_2, ERG_11, ERG_24, and the combination of constructs ERG_ALL). Gray circles represent untreated Pd control colonies. Pd colonies treated with high, medium, and low concentrations of dsRNA are depicted by orange, yellow and beige circles, respectively. Blue circles represent colonies treated with water, and green circles depict colonies treated with non-target dsRNA derived from the GUS gene in E. coli.

FIG. 5 shows photographs of colonies of Pd after single treatment with high, medium, and low concentrations of ERG1_1, ERG1_2, ERG_11, ERG_24, and ERG_ALL (combination of constructs).

FIG. 6A shows that drops of ERG11 dsRNA applied by pipette remain suspended on top of fungal colonies and FIG. 6B shows that the morphology of fungal mycelia in areas directly under these drops appears altered by the treatment. Under close observation these areas appear to be desiccated. No morphological changes are apparent in mycelia in control colonies (FIG. 6B).

FIG. 7 compares the number of conidia counted in control colonies (three replicates) and ERG11 treated colonies (three replicates). Points represent all the counted conidia in the total replicates.

FIGS. 8A-8C compare fungal area controlled by ERG_1_1 applied at three different treatment times after Pd inoculation: 7 days (FIG. 8A), 21 days (FIG. 8B), and 35 days (FIG. 8C). In this and subsequent FIGS. 9-12, lines are a nonparametric spline indicate the number of doses (Control, GUS, 1 dose, 2 doses, 3 doses, 4 doses, and 5 doses) used for the treatment of the colonies. Points represent the observations through time from first dose applied to 14 weeks later. They are plotted with jittering around their true value so that each point is visible. GUS dsRNA treatments had no inhibitory effect, indicating that target specific RNAi triggered inhibitory responses.

FIGS. 9A-9C compare fungal area controlled by ERG_1_2 applied at three different treatment times after Pd inoculation: 7 days (FIG. 9A), 21 days (FIG. 9B), and 35 days (FIG. 9C).

FIGS. 10A-10C compare fungal area controlled by ERG_11 applied at three different treatment times after Pd inoculation: 7 days (FIG. 10A), 21 days (FIG. 10B), and 35 days (FIG. 10C).

FIGS. 11A-11C compare fungal area controlled by ERG_24 applied at three different treatment times after Pd inoculation: 7 days (FIG. 11A), 21 days (FIG. 11B), and 35 days (FIG. 11C).

FIGS. 12A-12C compare fungal area controlled by ERG_ALL applied at three different treatment times after Pd inoculation: 7 days (FIG. 12A), 21 days (FIG. 12B), and 35 days (FIG. 12C).

FIGS. 13A-13E are boxplots comparing distribution of fungal area controlled by all targets when first dose was applied 7 days after Pd inoculation: ERG1_1 (FIG. 13A), ERG1_2 (FIG. 13B), ERG_11 (FIG. 13C), ERG_24 (FIG. 13D), and ERG_ALL (FIG. 13E).

FIGS. 14A-14E are boxplots comparing distribution of fungal area controlled by all targets when first dose was applied 21 days after Pd inoculation: ERG1_1 (FIG. 14A), ERG1_2 (FIG. 14B), ERG_11 (FIG. 14C), ERG_24 (FIG. 14D), and ERG_ALL (FIG. 14E).

FIGS. 15A-15E are boxplots comparing distribution of fungal area controlled by all targets when first dose was applied 35 days after Pd inoculation: ERG1_1 (FIG. 15A), ERG1_2 (FIG. 15B), ERG_11 (FIG. 15C), ERG_24 (FIG. 15D), and ERG_ALL (FIG. 15E).

FIGS. 16A-16E compare Pd viability after repeated dsRNA applications. To determine if ERG treatments rendered Pd inviable, Pd growth was monitored for 60 days after 1, 2, 3, 4, or 5 repeat treatments, with the first treatment applied on day 7, 14, or 28 after fungal plating. 5 μL of the treated colonies was then replated. If growth was not seen in replated colonies after 90 days, it was concluded that a dsRNA treatment had rendered Pd inviable. Growth inhibition was observed in all replated colonies. Three dsRNA treatments (ERG1_1, ERG1_2, and ERG_24) rendered Pd inviable. Colonies treated with ERG1_2 dsRNA were rendered inviable after two doses. Reductions in colony regrowth were statistically significant versus the control in all treatments (p<0.05), apart from colonies treated once with ERG1_1 before harvesting.

FIGS. 17A and 17B compare the average absorbance (AO) values over time for Pd cultures treated with three once-weekly doses of dsRNA targeting the chitin biosynthesis pathway. Treatments included CHS2_1, CHS2_2, and a combination of both (CHS_combo), each applied at high (5 μg/μL) (FIG. 17A) and low (1 μg/μL) (FIG. 17B) concentrations. Control wells received water only. Absorbance was measured weekly to monitor Pd growth inhibition. CHS_combo at high concentration showed the most pronounced inhibition after three doses, with reduced growth compared to controls, which plateaued around day 50.

FIGS. 18A and 18B compare the average absorbance (AO) values over time for Pd cultures treated with five once-weekly doses of CHS2_1, CHS2_2, and CHS_combo dsRNA. Both high (5 μg/μL) (FIG. 18A) and low (1 μg/μL) (FIG. 18B) concentrations were tested. Absorbance was monitored weekly and again at two and three months post-treatment to assess long-term inhibition. CHS2_1 was the most effective treatment, with Pd growth plateauing around day 28, significantly earlier than the control group 2.

### DETAILED DESCRIPTION

The present disclosure provides a novel approach for the control of Pseudogymnoascus destructans (Pd). The active ingredient is a nucleic acid—a double-stranded RNA (dsRNA)—that can be used as a fungicidal formulation, for example, as a spray. The sequence of the dsRNA corresponds to a part or the whole of an essential fungal gene and causes downregulation of the fungal target via RNA interference (RNAi). As a result of the downregulation of mRNA, the dsRNA prevents expression of the target fungal protein and hence causes death, growth arrest or nonviability of the fungus on substrates and/or within bat tissues.

Bats are susceptible to myriad viral infections including deadly zoonoses such as rabies and distemper. North American bats have been exposed to these pathogens generationally, which enabled these species to develop immunity. Because Pd is a novel fungal pathogen only recently introduced to the continent, North American bats have no inborn immunity to fungal infections and this intolerance has led to mortalities in significant enough numbers to cause colonies of hibernating bats to collapse and drive multiple species to the brink of extinction. The present disclosure provides methods and compositions to reduce Pd load in hibernacula substrates and to control Pd infection and Pd loads in bat tissues.

RNA interference refers to the process of sequence-specific post-transcriptional gene silencing in animals mediated by small RNAs. The corresponding process in plants is commonly referred to as “post-transcriptional gene silencing” or “RNA silencing” and is also referred to as “quelling” in fungi. While not being limited to any particular theory, the process of post-transcriptional gene silencing is thought to be an evolutionarily conserved cellular defense mechanism used to prevent the expression of foreign genes and a mechanism for gene regulation. It is commonly shared by diverse taxa. Such protection from foreign gene expression may have evolved in response to the production of double-stranded RNAs (dsRNAs) derived from viral infection or from the random integration of transposon elements into a host genome via a cellular response that specifically destroys homologous single-stranded RNA or viral genomic RNA. In aspects according to the present disclosure, a nucleic acid composition results in RNA interference in a target organism. In certain aspects, the nucleic acid composition results in RNA interference in Pseudogymnoascus destructans when applied to and taken up by fungal cells.

Pd is detected in the environment and on bats by sampling substrates or tissues and conducting an assay sensitive enough to signal Pd presence using real time polymerase chain reaction (qPCR). The presence of a Pd infection in bats can also be perceived visually due to its obvious white, filamentous appearance. Reservoirs of the fungus include both substrates—caves, sediments, guano, bat boxes, bridges, etc.—and bats, including individual bats and bat colonies.

The methods of the invention can find practical application in any area of technology where it is desirable to inhibit viability, growth, development, or sporulation of Pd, or to decrease the reservoir of the fungus. The methods of the invention further find practical application where it is desirable to specifically down-regulate expression of one or more target genes in Pd.

Particularly useful practical applications include, but are not limited to, (1) reducing the reservoir of Pd in caves and hibernacula, including manmade structures known to house hibernating bats; (2) preventing the spread or establishment of Pd colonies within caves and hibernacula, including manmade structures known to house hibernating bats; and (3) use on bats to control, treat or prevent Pd infections.

In accordance with one embodiment the invention relates to a method for controlling Pd growth on a substrate, cell or an organism, or for preventing establishment of Pd colonies on substrates, within a cell or on an organism susceptible to Pd infection, comprising contacting Pd structures—hyphae, mycelia, spores—with a double-stranded RNA, wherein the double-stranded RNA comprises annealed complementary strands, one of which has a nucleotide sequence which is complementary to at least part of the nucleotide sequence of a Pd target gene, whereby the double-stranded RNA is taken up by the fungus and thereby controls growth, reduces fungal load, prevents the establishment of fungal colonies, or renders the fungus nonviable.

The present invention therefore provides isolated novel nucleotide sequences of fungal target genes, said isolated nucleotide sequences comprising at least one nucleic acid sequence selected from the group comprising genes related to the production of ergosterol, a substance necessary for the integrity fungal cell membranes. Exemplary genes include but are not limited to squalene epoxidase (ERG_1 and ERG_2), lanosterol 14-alpha-demethylase (ERG11), and sterol reductase (ERG24).

In another embodiment, a gene is selected that is essentially involved in the synthesis of chitin, a substance necessary for the integrity of fungal cell walls. Exemplary genes include but are not limited to chitin synthase 2 (CHS2_1 and CHS2_2), and an additional chitin synthase class gene (CHS3). Similar sequences have been found in diverse organisms such as Fusarium graminearum, Phoma lingam, and Ustilago maydis. Related sequences are found in diverse organisms such as Kalanchoe fedteschenkoi, and Cephalotus follicularis.

Other target genes for use in the present invention may include, for example, those that play important roles in viability, growth, development, reproduction, sporulation, and virulence. These target genes include, for example, housekeeping genes, transcription factors, and fungus-specific genes or lethal knockout mutations in Pd. The target genes for use in the present invention may also be those that are from other organisms, e.g., from Pseudogymnoascus spp. or other filamentous fungi (e.g., Fusarium spp.).

Selected Pd housekeeping genes—in one embodiment, ergosterol biosynthesis genes associated with membrane protein production and in another embodiment, chitin synthase genes associated with cell wall structures—were analyzed and dsRNA constructs were designed around subregions of these genes to maximize the production of small interfering RNAs (siRNA) and promote robust RNAi gene silencing. Manufactured dsRNA molecules targeting these subregions are applied externally to individual Pd cells or entire Pd colonies. Exogenous dsRNA is manufactured using techniques such as in vitro transcription, cell-free systems for in vitro protein expression (also referred to as in vitro translation, or cell-free protein expression), and production in microbial or fungal systems (Hough et al. 2022).

Exogenously applied RNAi treatments can consist of naked dsRNA or dsRNA bound to particles or nanocarriers that are suspended in aqueous or other solutions. Particles bound to dsRNA to improve molecule stability include but are not limited to poly (butylene terephthalate) (PBT) nanocomposites (Berti et al. 2009), chitosan, carbon dots, silica nanoparticles (Das et al. 2015), montmorillonite, kaolinite (Gallori et al. 1996), montmorillonite nanoclays (Gujjari et al. 2018), chitosan nanoparticles (Kumar et al. 2016), layered double hydroxide (LDH) nanoparticles (Malla et al. 2023), liposomes (Lin et al. 2017), halloysite nanotubes (Liu et al. 2019), lipofectamine (Nami et al. 2017), nanoclays (Pal et al. 2023), inorganic nanoparticles (Sokolova and Epple 2008), peptides and polymers (Yang et al. 2022).

dsRNA constructs in accordance with the present disclosure effectively control Pd growth and render Pd inviable. As indicated above, these dsRNA molecules may be bound to a particle or nanoparticle, and this particle suspended in a sprayable and/or aerosolized solution. This solution may be applied using industrial sprayers at sites where bats roost and Pd is known to exist such as caves, bat boxes, culverts, and bridges.

As noted above, the present invention provides methods and compositions for RNAi-mediated control of Pseudogymnoascus destructans. In particular, the present invention provides double-stranded ribonucleic acid (dsRNA) constructs, as well as related compositions and methods, for controlling Pd growth and viability by repressing, delaying, or otherwise reducing gene expression within Pd.

In one aspect, the present invention provides a double-stranded ribonucleic acid (dsRNA) for inhibiting expression of a target gene in Pseudogymnoascus destructans, wherein the target gene is selected from a gene involved in the synthesis of ergosterol and a gene involved in the synthesis of chitin, and wherein the dsRNA includes a first strand comprising a region of complementarity that is substantially complementary to a target region of the mRNA encoded by the target gene. In some embodiments, the region of complementarity is from 85% to 100% complementary with the mRNA target region. In some embodiments, a second strand of the dsRNA is complementary to the first strand. The region of complementarity may be, for example, at least 20 nucleotides in length, at least 25 nucleotides in length, or at least 30 nucleotides in length. In some variations, the RNA sequence of the first strand is at least 100 nucleotides in length (e.g., from 100 to 500 nucleotides in length).

In certain embodiments of a dsRNA as above wherein the target gene is involved in the synthesis of ergosterol, the target gene is selected from ERG1_1, ERG1_2, ERG_11, and ERG_24. In some such variations, the mRNA target region is encoded by a target gene sequence contained within, comprising, or consisting of the sequence shown in SEQ ID NO:2, SEQ ID NO:4, SEQ ID NO:6, or SEQ ID NO:8. Suitable first strand RNA sequences targeting ERG1_1, ERG1_2, ERG_11, or ERG_24 include (a) a first strand RNA sequence contained within the sequence shown in SEQ ID NO:18, SEQ ID NO:20, SEQ ID NO:22, or SEQ ID NO:24; (b) a first strand RNA sequence comprising a sequence having from 95% to 100% identity to the sequence shown in SEQ ID NO:18, SEQ ID NO:20, SEQ ID NO:22, or SEQ ID NO:24 (e.g., a sequence comprising the sequence shown in SEQ ID NO:18, SEQ ID NO:20, SEQ ID NO:22, or SEQ ID NO:24; and (c) a first strand RNA sequence consisting of a sequence having from 95% to 100% identity to the sequence shown in SEQ ID NO:18, SEQ ID NO:20, SEQ ID NO:22, or SEQ ID NO:24 (e.g., a sequence consisting of the sequence shown in SEQ ID NO:18, SEQ ID NO:20, SEQ ID NO:22, or SEQ ID NO:24).

In certain embodiments of a dsRNA as above wherein the target gene is involved in the synthesis of chitin, the target gene is selected from CHS2_1, CHS2_2, and CHS3. In some such variations, the mRNA target region is encoded by a target gene sequence contained within, comprising, or consisting of the sequence shown in SEQ ID NO:10, SEQ ID NO:12, or SEQ ID NO:14. Suitable first strand RNA sequences targeting CHS2_1, CHS2_2, or CHS3 include (a) a first strand RNA sequence contained within the sequence shown in SEQ ID NO:26, SEQ ID NO:28, or SEQ ID NO:30; (b) a first strand RNA sequence comprising a sequence having from 95% to 100% identity to the sequence shown in SEQ ID NO:26, SEQ ID NO:28, or SEQ ID NO:30 (e.g., a sequence comprising the sequence shown in SEQ ID NO:26, SEQ ID NO:28, or SEQ ID NO:30; and (c) a first strand RNA sequence consisting of a sequence having from 95% to 100% identity to the sequence shown in SEQ ID NO:26, SEQ ID NO:28, or SEQ ID NO:30 (e.g., a sequence consisting of the sequence shown in SEQ ID NO:26, SEQ ID NO:28, or SEQ ID NO:30).

In certain variations of a dsRNA as above, the dsRNA includes at least one modified nucleotide. Particularly suitable modified nucleotides include nucleotides comprising a 2′-O-methyl modified nucleotide and nucleotides comprising a 5′-phosphorothioate group. In other embodiments, a modified nucleotide is selected from a 2′-deoxy-2′-fluoro modified nucleotide, a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, and a non-natural-base-comprising nucleotide.

In a related aspect, the present invention provides a composition for controlling Pseudogymnoascus destructans, wherein the composition comprises a dsRNA as above. In some embodiments, the dsRNA is naked dsRNA. In other embodiments, the dsRNA is bound to a carrier to improve stability and/or delivery of the dsRNA. In certain variations, the carrier is a particle or nanocarrier. In some embodiments, the carrier is selected from the group consisting of a poly (butylene terephthalate) (PBT) nanocomposite, chitosan, a carbon dot, a silica nanoparticle, montmorillonite, kaolinite, a chitosan nanoparticle, a layered double hydroxide (LDH) nanoparticle, a liposome, a halloysite nanotube, lipofectamine, a nanoclay, an inorganic nanoparticle, a peptide, and a polymer. The composition may be formulated for administration by, for example, spraying or fogging. In certain variations, the composition is an aerosolized formulation. In other, non-mutually exclusive embodiments, the composition is an aqueous formulation. In certain variations, a composition as above comprises two or more dsRNAs for inhibiting expression of two or more Pseudogymnoascus destructans target genes.

In another aspect, the present invention provides a method for controlling Pseudogymnoascus destructans (Pd) in the environment. The method generally includes administering to a substrate comprising Pd, or to a substrate susceptible to establishment of Pd, an effective amount of a dsRNA as described above. In some embodiments, the dsRNA is naked dsRNA. In other embodiments, the dsRNA is bound to a carrier to improve stability and/or delivery of the dsRNA. In certain variations, the carrier is a particle or nanocarrier. In some embodiments, the carrier is selected from the group consisting of a poly (butylene terephthalate) (PBT) nanocomposite, chitosan, a carbon dot, a silica nanoparticle, montmorillonite, kaolinite, a chitosan nanoparticle, a layered double hydroxide (LDH) nanoparticle, a liposome, a halloysite nanotube, lipofectamine, a nanoclay, an inorganic nanoparticle, a peptide, and a polymer. The dsRNA may be administered, for example, by spraying or fogging. In certain variations, the dsRNA is contained in an aerosolized formulation. In other, non-mutually exclusive embodiments, the dsRNA is contained in an aqueous formulation. In certain variations, a method as above comprises administering two or more dsRNAs for inhibiting expression of two or more Pd target genes.

A method for controlling Pd in the environment as above may include a single or multiple administrations of the dsRNA. In some variations, the dsRNA is administered to a substrate comprising Pd hyphae and/or Pd spores. In other, non-mutually exclusive embodiments, the substrate is a bat hibernacula substrate such as, for example, a cave or a manmade structure known to house hibernating bats (e.g., a manmade structure is selected from a bridge, a mine shaft, and a bat box). In some embodiments wherein the substrate is a bat hibernacula substrate, the method reduces the reservoir of Pd on or within the bat hibernacula substrate; in other embodiments, the method prevents the establishment of Pd on or within the bat hibernacula substrate. In certain embodiments wherein the substrate is a bat hibernacula substrate, the method treats or prevents Pd infections in bats.

In another aspect, the present invention provides a method for treating or preventing white-nose syndrome in a bat. The method generally includes administering to a bat having or at risk of developing white nose syndrome an effective amount of a dsRNA as above. In some embodiments, the dsRNA is naked dsRNA. In other embodiments, the dsRNA is bound to a carrier to improve stability and/or delivery of the dsRNA. In certain variations, the carrier is a particle or nanocarrier. In some embodiments, the carrier is selected from the group consisting of a poly (butylene terephthalate) (PBT) nanocomposite, chitosan, a carbon dot, a silica nanoparticle, montmorillonite, kaolinite, a chitosan nanoparticle, a layered double hydroxide (LDH) nanoparticle, a liposome, a halloysite nanotube, lipofectamine, a nanoclay, an inorganic nanoparticle, a peptide, and a polymer. The dsRNA may be administered, for example, by spraying or fogging. In certain variations, the dsRNA is contained in an aerosolized formulation. In other, non-mutually exclusive embodiments, the dsRNA is contained in an aqueous formulation. In still other, non-mutually exclusive embodiments, the dsRNA is formulated for transdermal delivery. In certain variations, a method as above comprises administering two or more dsRNAs for inhibiting expression of two or more Pd target genes. Administration of the dsRNA may include single or multiple administrations.

The following describes representative methods and compositions for the control of Pseudogymnoascus destructans (Pd), the active ingredient is a nucleic acid—a double-stranded RNA (dsRNA)—that can be used as a fungicidal formulation.

### Bioinformatic Target Identification

Using the annotated Pd genome (Drees et al. 2016) as a reference, the presence of certain genes implicated in RNAi and related RNA silencing phenomena—namely QDE1, QDE2, QDE3, RDRP, Dicer, and Argonaut (Ago1, Ago2)—was verified in Pd (Dudley et al. 2005; Table 1) to confirm the biological potential to trigger an RNAi response.

TABLE 1
Proteins implicated in RNAi and
related RNA silencing phenomena.

| | C. elegans | Drosophila | Humans | Plants | Fungi |
|---|---|---|---|---|---|
| Dicer | DCR-1 | Dicer | Dicer | CAF/ | Dicer |
| RNase | | | | Sin-1 | |
| RNA- | EGO-1, | | | SGS2/ | QDE1, |
| dependent | RRF-1, | | | SDE1 | RDRP, |
| RNA | RRF-3 | | | | RrpA |
| polymer- | | | | | |
| ases | | | | | |
| Proteins | RDE-1 | AGO2, | elF2C1/2 | AGO1, | QDE2, |
| with | | Piwi, | | AGO4 | Ago1, |
| PAZ/piwi | | Aubergine | | | Ago2 |
| domains | | | | | |
| Nucleases | MUT-7 | Tudor-SN | | WEX-1 | |
| Helicases | MUT-14, | p68, | | MUT6, | QDE3 |
| | DRH-1/2 | Spindle-E | | SDE3 | |
| Chromatin | MES-3, -4, -6 | | | DDM1, | |
| modifiers | | | | MET1 | |
| dsRNA- | RDE-4 | R2D2 | | | |
| binding | | | | | |
| protein | | | | | |
| Nonsense- | SMG2, | | | | |
| mediated | SMG-5, | | | | |
| decay | SMG-6 | | | | |
| Other | SID-1 | FMRP, | | SGS3, | |
| proteins | | dFXR, | | HEN1 | |
| | | VIG | | | |

Genes typically associated with antifungal properties (Mazu et al. 2016, Bhattacharya et al. 2018) were identified as potential RNAi targets. To identify and design dsRNA constructs, genes in the ergosterol biosynthesis (ERG1_1, ERG1_2, ERG11, and ERG24) and chitin synthase pathways (CH3, CHS2_1, and CHS2_2) were targeted, and si-Fi (Lück et al. 2019) was used to maximize siRNA presence within regions between 200 and 300 bases in length within these genes. Treatments are derived from and associated with any part of the DNA nucleotide sequences listed below, as well as their reverse complements, the RNA transcripts from either forward or reverse DNA strand, their associated translated proteins, and RNA and DNA nucleotide sequences capable of producing these associated proteins. The sequences of each gene and its associated dsRNA construct are shown in SEQ ID NO:1-8 (Sequences Associated with Ergosterol Biosynthesis) and SEQ ID NO:9-14 (Sequences Associated with Chitin Synthase).

All sequences for all genes were evaluated in silico using genomes of nontarget species as described in Examples 1 and 2 to select regions that minimized off target effects and maximized siRNA production to trigger robust RNAi gene silencing in Pd.

To verify that observed growth inhibition was caused by dsRNA targets as opposed to the introduction of any dsRNA to Pd cells, a dsRNA control was designed based on the beta-glucuronidase (Beta-D-glucuronosideglucuronosohydrolase) (GUS) gene in Escherichia coli (E. Coli) using the methods described above for identifying dsRNA targets for Pd. The sequences of the GUS gene and its associated dsRNA construct shown in SEQ ID NO:15 and 16 (Sequences Associated with GUS).

### Verification of dsRNA Uptake by Pd

Uptake of dsRNA and gene silencing requires the presence of various proteins (see Table 1) and can be affected by dsRNA molecule length, presence of nucleases, fungal surface, and other factors (Šečić and Kogel 2021). Prior to treating Pd with dsRNA constructs, the following laboratory experiments were performed to verify dsRNA uptake in Pd cells and confirm the presence of RNAi mechanisms in the fungus.

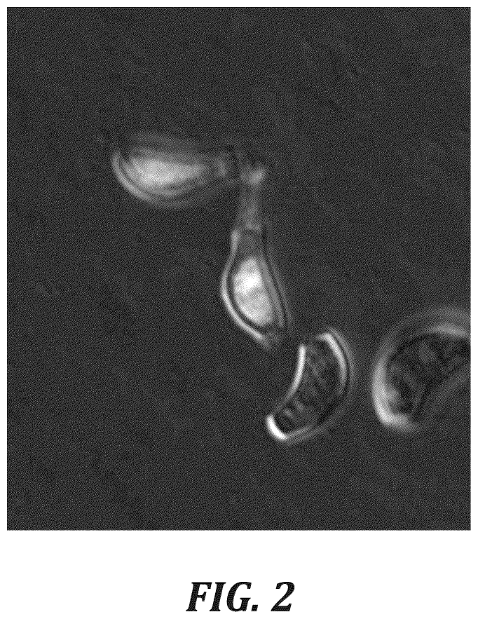

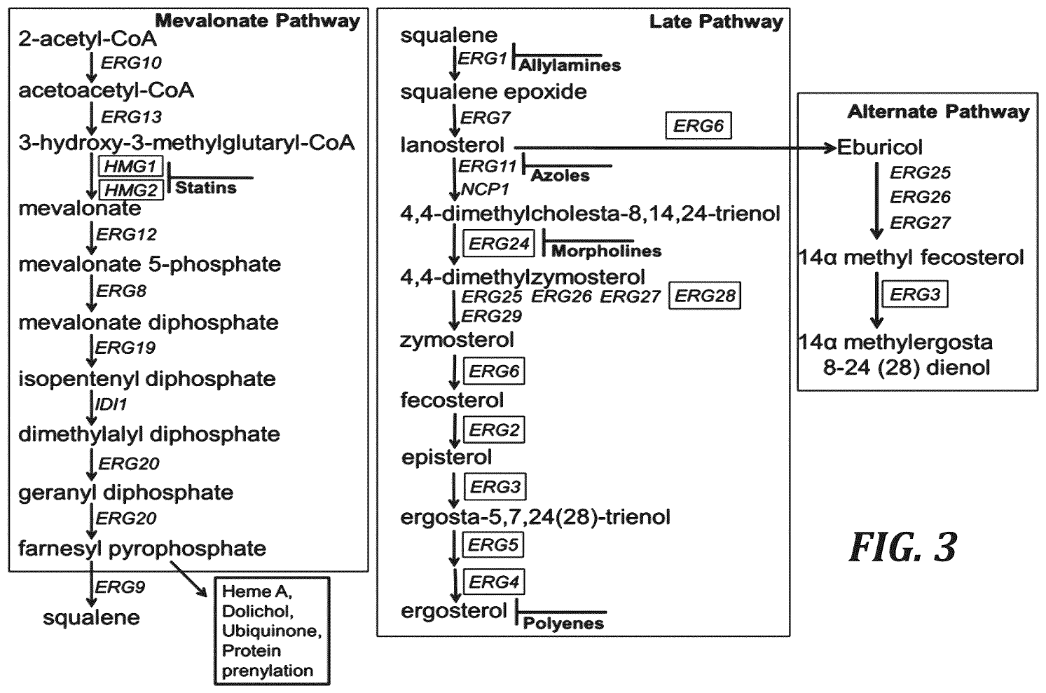

To evaluate dsRNA uptake by Pd spores, fluorescently labeled dsRNA were applied to germinating fungal cells. A Pd solution (4 μL of dsRNA combined with about 300 conidia) was prepared and deposited it to a thin layer of Sabouraud dextrosa agar (SDA) on a glass slide to ensure the germination process was not disrupted. Glass slides were then incubated inside a petri dish at 7° C. for 48 hours. This material was examined under a Zeiss LSM 780 NLO confocal microscope and photographed the results. The presence of fluorescence in Pd cells confirms Pd uptake of dsRNA constructs (FIG. 2).

### Pd Growth Inhibition by RNAi Gene Silencing of Ergosterol Biosynthesis and Chitin Synthase

Once dsRNA uptake was demonstrated, as an initial evaluation of whether the dsRNA targets would trigger RNAi, two experiments were first initiated using a single dose of each target prior to germination and on actively growing Pd to determine both whether there was any evidence of inhibition and how this varied by dose. Post-germination experiments were then conducted, beginning at different stages of fungal growth and with variable number of weekly doses to evaluate the requirements for repeat treatments and the consistency of the response at different stages of fungal growth.

### Single Dose Pre-Germination Experiment

The inhibitory effects of dsRNA targets on Pd growth were evaluated using in vitro 96 well plate microassays and SDA petri dish replicates. Suspensions of 50 μl (about 100 conidia/μl) were prepared in 96 well plates with Saboraud nutrient broth. 96 well plates and petri dishes were inoculated at 7° C. during the experiment.

To determine if an RNAi control targeting the ergosterol biosynthesis (ERG) pathway effectively inhibited Pd growth, fungal spores and individual ERG dsRNA treatments were combined in 96 well plates. High (0.33 μg/μL), medium (0.16 μg/μL), and low (0.016 μg/μL) concentrations of five treatments (ERG1_1, ERG1_2, ERG11, ERG24, and all constructs combined (ERG_ALL) were evaluated by mixing 50 μL of dsRNA with conidia spores (50 μL) before germination.

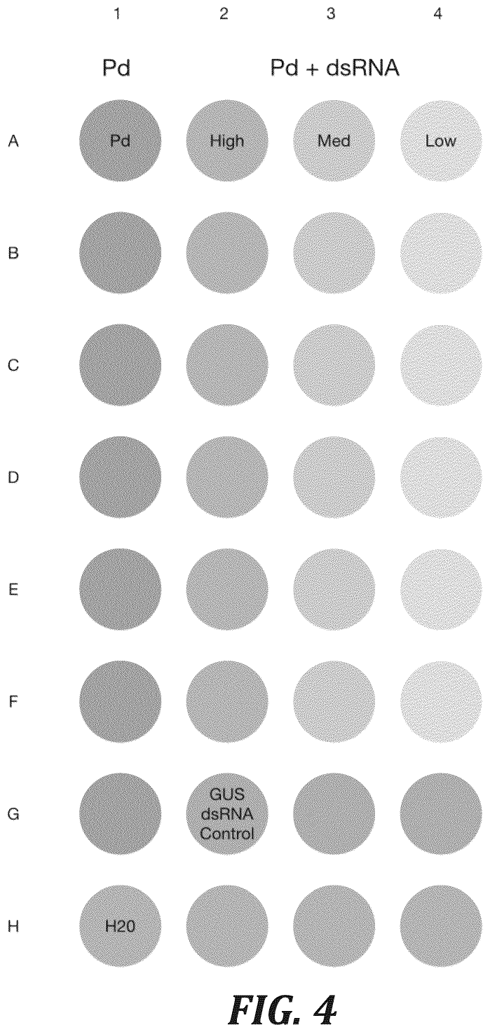

The treatments were assigned as follows: Column 1 wells A-G were inoculated with Pd alone, columns 2, 3, and 4 were inoculated with a combination of Pd and high, medium, and low dsRNA concentration respectively in wells A-F, and wells in row G were inoculated with Pd and negative control (GUS). All wells in row H were inoculated with water alone. (FIG. 4).

### Single Dose Pre-Germination Results

Treated colonies were observed to evaluate the effects of exogenously applied dsRNA ERG suspensions on Pd. Observed changes included irregular borders and desiccated mycelia. No differences were found in the borders of colonies that had been treated with either dsRNA ERG11 or ERG24 (see Table 2).

TABLE 2
Characteristics of fungal colonies
after being treated with ERG dsRNA.

| | Irregular | Dry | |
|---|---|---|---|
| Treatment | Borders | Colonies | Hydrophobicity |
| ERG 1_1 | x | x | x |
| ERG 1_2 | x | x | x |
| ERG 11 | | | x |
| ERG 24 | | x | x |
| ERG ALL | x | x | x |

For all ERG treatments, some colonies did not appear to readily absorb exogenously applied dsRNA when applied pre-germination. Treatments were delivered via pipettor and formed a droplet on the surface of fungal mycelia. This suggests that treatment uptake by fungal colonies could be improved if dsRNA molecules are bound to particles that facilitate entry into hydrophobic fungal cells.

### Single Dose Post-Germination Experiment

Following the same layout used for the microassays and petri plates in the pre-germination experiment, Pd colonies were allowed to grow for a period four days and evaluated the effects of previously mentioned ERG dsRNA molecules (ERG1_1, ERG1_2, ERG11, ERG24, and the combination of all dsRNA constructs (ERG_ALL), on germinated spores.

### Single Dose Post-Germination Results

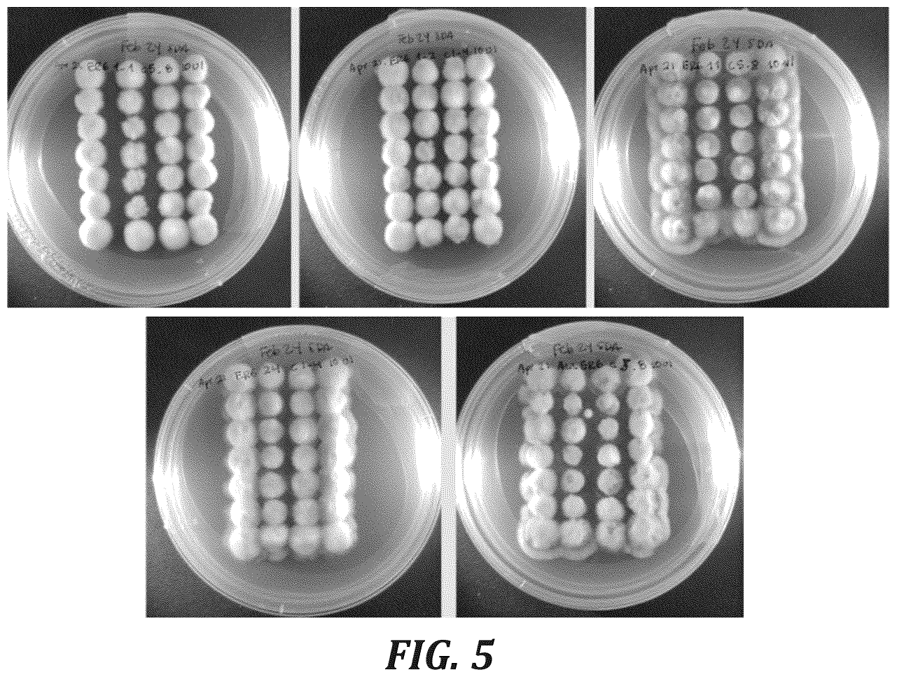

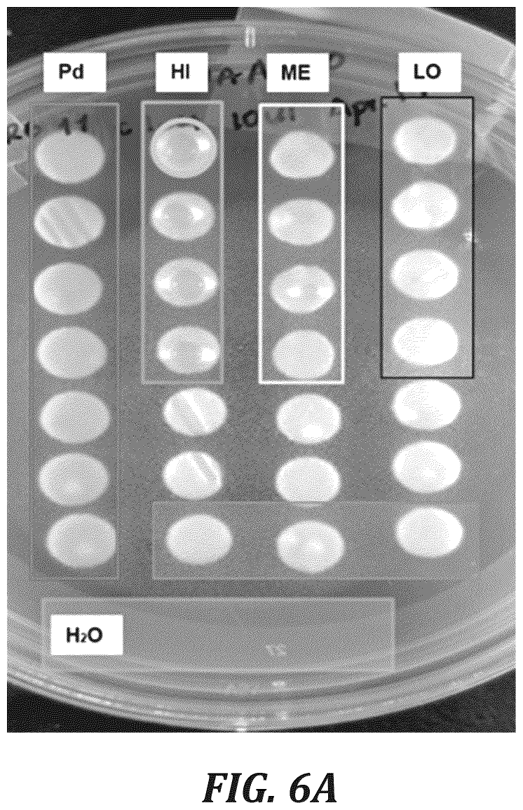

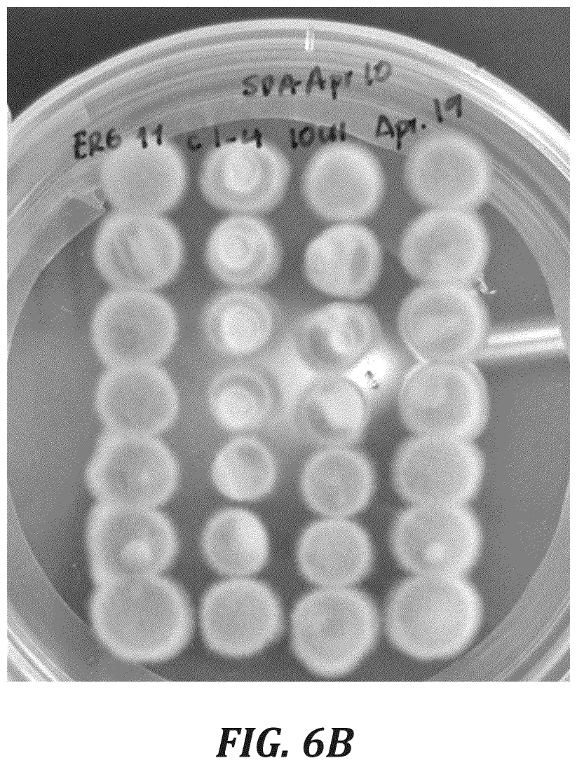

Experiments in vitro demonstrated that dsRNA treatments applied after spore germination effectively inhibited fungal growth. Decreased mycelial height was observed in colonies treated with high- and medium-concentration dsRNA. The same results were observed in all targets except for one of the three ERG1_2 replicates. There was no change in mycelial height when colonies were treated with low concentration dsRNA (FIG. 5).

### Increasing Concentration Dose For Pre-Germination Experiment Colonies

After 16 days, few changes were observed in the colonies treated pre-germination and retreated colonies with an additional 20 μL of high (1 μg/μL), medium (0.50 μg/μL), and low (0.25 μg/μL) dsRNA concentrations. Changes in colonies were evaluated over a period of 48 days. The experimental layout was similar to the pre-germination experiment (FIG. 5); however, treatments were applied only to the first four rows of colonies.

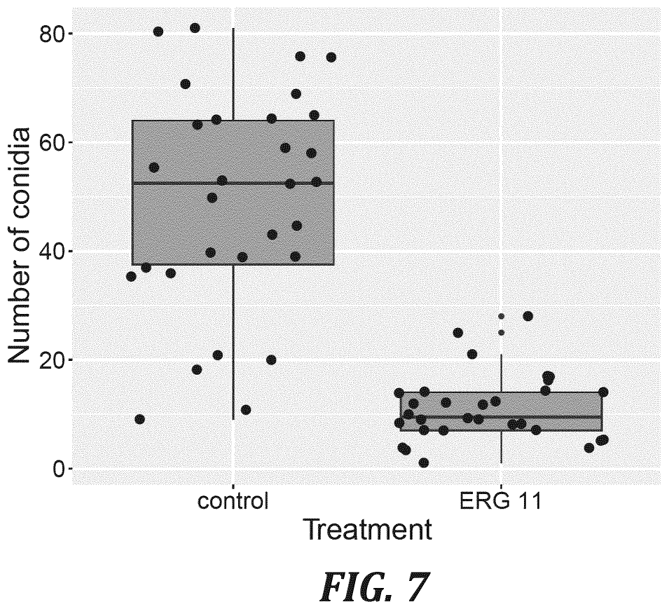

No mycelial growth was observed in colony areas touching high concentration (1 μg/μL) ERG1_1, ERG1_2 and ERG11 dsRNA solutions. For medium concentration (0.50 μg/μL) solutions, colonies treated with ERG1_2 and ERG11 exhibited reduction of fungal growth. In colonies treated with all concentrations of ERG24 and ERG_ALL, growth continued through the applied droplet and on the periphery of the colonies. At the end of the experiment, to evaluate changes in conidia production, one control colony and one treated colony of the three replicates treated with high concentration ERG11 were harvested. Treated colonies produced fewer conidia (mean 11.06±SD 6.28) than control colonies (mean 49.4±SD 20.30). (FIG. 7)

### Repeated Dose Experiment

Once the presence of the RNAi mechanism in Pd was verified and a strong inhibitory response was established in the single dose post-germination experiment, the effects of repeated applications of dsRNA treatments on Pd growth were quantified while varying the timing of the initial treatment to ensure that treatments would be robust to variable timing within the fungal life cycle. Fungal colonies were sprayed starting 7 (dose type 1), 21 (dose type 2), and 35 days (dose type 3) after Pd plating with 20 μL (1 μg/μL) dsRNA suspension. This constituted the timing of the first dsRNA treatment, and subsequently four fungal colonies per treatment event were exposed to a specific dsRNA construct. Each of these replicates of four colonies received one to five treatment doses. Fungal colonies were photographed weekly for at least three months to document Pd growth over time. At each timestamp, to track detected changes in colony growth, a binary scoring system was employed using a value of 1 to denote that an inhibitory effect was observed, and 0 if not. In addition, to quantify growth, mycelial coverage was tracked and a percentage value (0, 25, 50, 75 or 100%) was assigned to describe the amount of fungal area controlled by dsRNA treatments with a value of 100% denoting total control of mycelia growth. Finally, a categorical variable was used to describe colony dryness, assigning 0 if no change was observed, 1 if a colony was partially dry, and 2 to indicate complete desiccation. Using generalized linear models, how number of doses and days of growing predicted the area of fungal inhibition was examined.

At the end of the experiment, fungal colonies that had been treated four or five times were harvested and ergosterol production was quantified using High Performance Liquid Chromatography (HPLC).

Both controls and GUS treatments resulted in no fungal growth inhibition. All dsRNA treatments inhibited Pd growth showing a significant reduction in the fungal area versus GUS and control colonies with the extent of inhibition varying by dsRNA treatment, number of doses, and application timing (dose type).

The most effective treatments controlled up to 100 percent of fungal area across all colonies. While inhibition was similarly strong across the three initial dose timings, the strongest effect was observed when the initial application occurred after 14 days of growth. For all treatments, the mean area controlled by the fungus exceeds 50% when 2 or more doses are applied following 14 days of fungal growth.

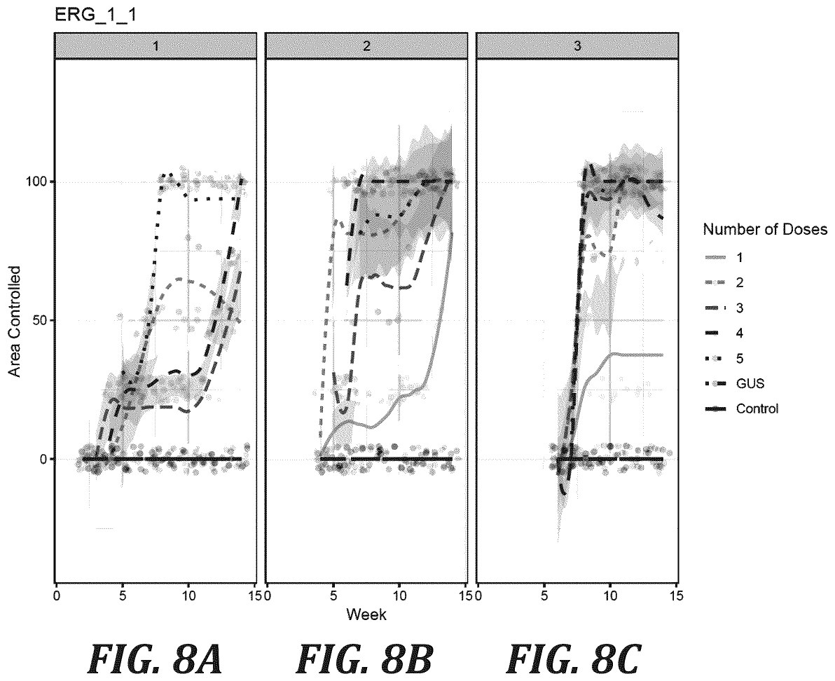

The inhibition through time for all targets, number of doses (1-5), and timing of initial dose (day 7, 21, 35) are summarized in FIGS. 8A-12C.

Pooling across all doses (1-5), Target ERG1_1 applied 7 days after Pd inoculation controlled an estimated 62.5% of colony area (SE 11.46, p=2×10⁻⁷) and 85% of the area when applied to colonies growing for 35 days (SE 7.43, p<2×10⁻¹⁶) (FIGS. 8A-8C). Dose type 2 applied 21 days after Pd plating controlled the highest percent of fungal area, (96% SE 7.008, p<2×10⁻¹⁶) (FIG. 8B) when ≥2 doses were applied.

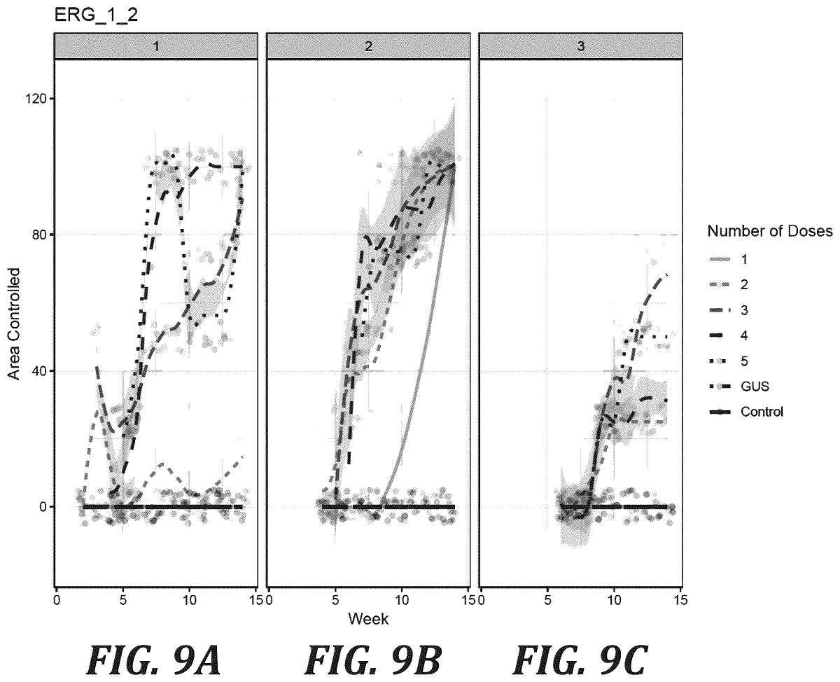

Similarly, ERG1_2 controlled 100% of fungal growth when treatment started 21 days after Pd inoculation (SE 7.008, p<2×10⁻¹⁶). Only 35% of fungal area was controlled when doses were applied 35 days after fungal inoculation (SE 7.432, p=6.17×10⁻⁶) and had no effect when only one dose was applied. Doses of ERG1_2 applied 14 days post inoculation controlled 61% of fungal area (SE 11.46, p=3.79×10⁻⁷) (FIGS. 9A-9C).

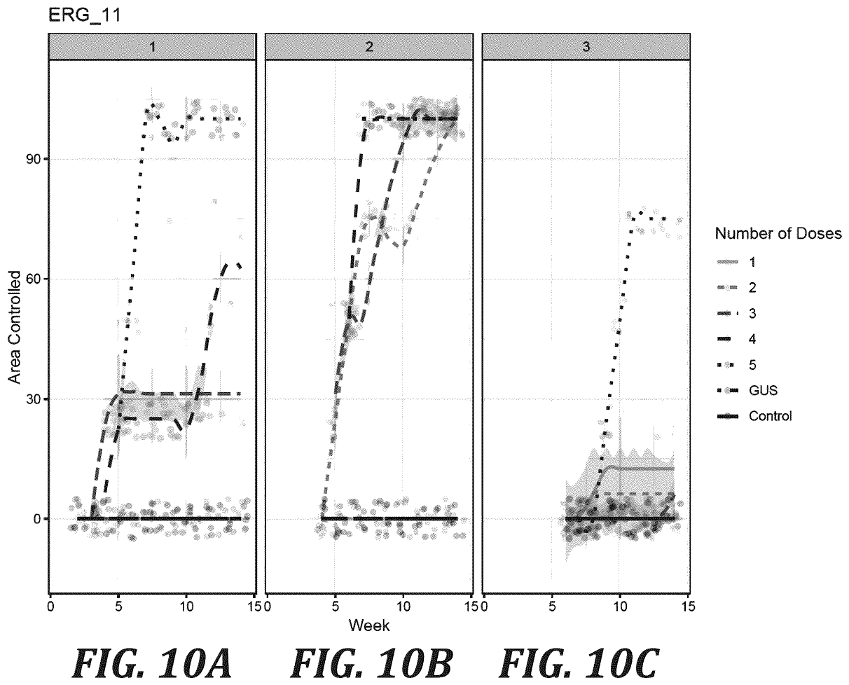

Fungal growth inhibition by ERG11 was highly variable. Doses applied 21 days after Pd inoculation controlled up to 80% of colony area (SE 7.008, p><2×10⁻¹⁶) with dose type 1 controlling 38% (SE 1.14, p=0.00094) and dose type 3 controlling 20% (SE 7.432, p=0.008) (FIGS. 10A-10C).

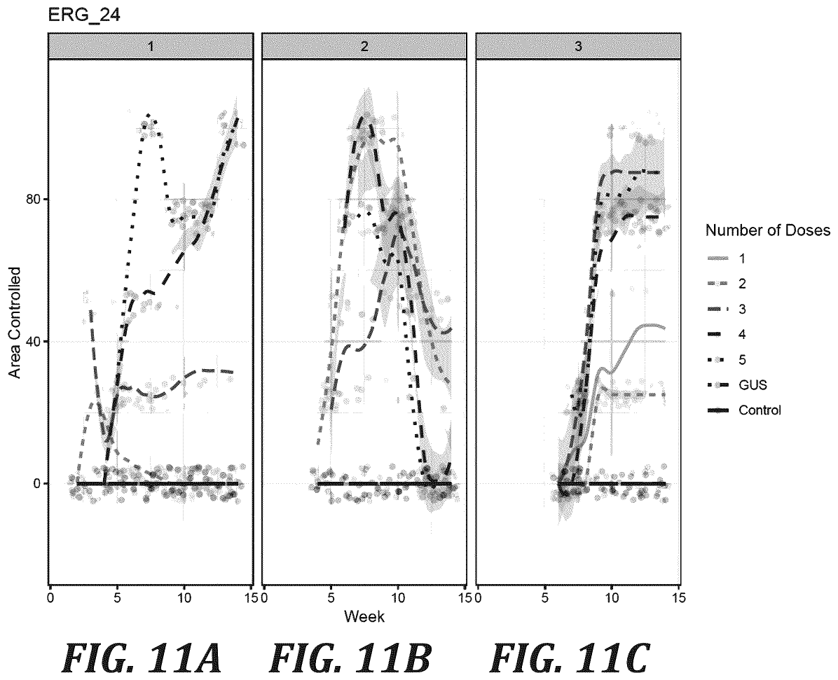

The area controlled by ERG24 ranged from 16% (SE 7.008, p=0.0219) in colonies dosed after 21 days to 63% (SE 7.432, p=220×10⁻¹⁴) for colonies dosed after 35 days. Colonies treated 21 days after inoculation regrew despite receiving repeated doses that inhibited fungal growth. For dose type 1, 46% of fungal area was controlled (SE 11.46, p=9.08×10⁻⁵) (FIGS. 11A-11C).

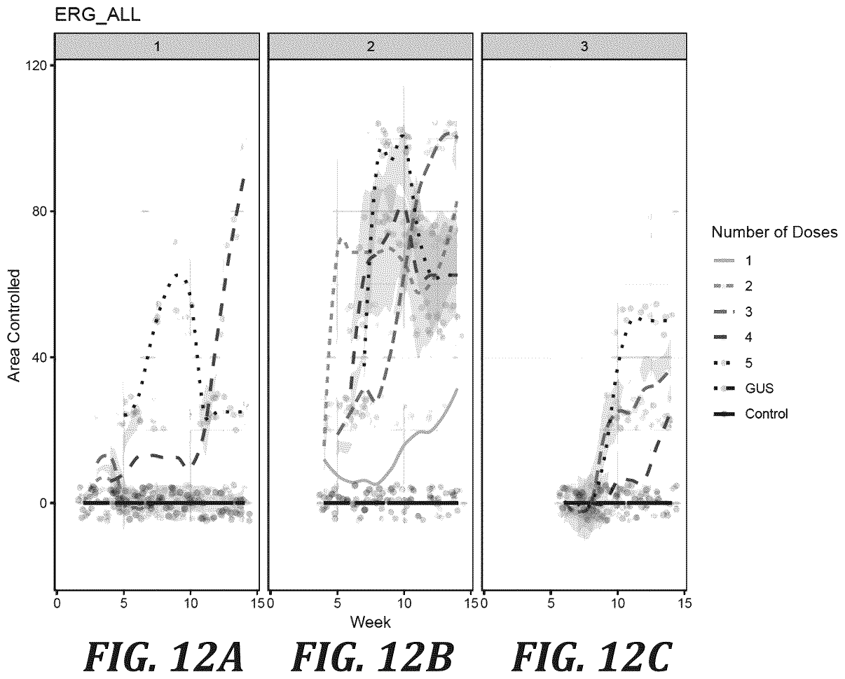

When using all targets in solution, ERG_ALL, 22% of colony area was controlled for colonies treated with dose type 1 (SE 11.46, p=0.051) and dose type 3 (SE 7.43, p=0.00296). A higher control of fungal growth is reported for dose type 2 with 67% control of colony area (SE 7.008, p<2×10⁻¹⁶) (FIGS. 12A-12C).

The effects of repeated dsRNA applications (1 μg/μL) on established fungal colonies were examined. Colonies received initial dsRNA inoculations after 7, 21, or 35 days of fungal growth.

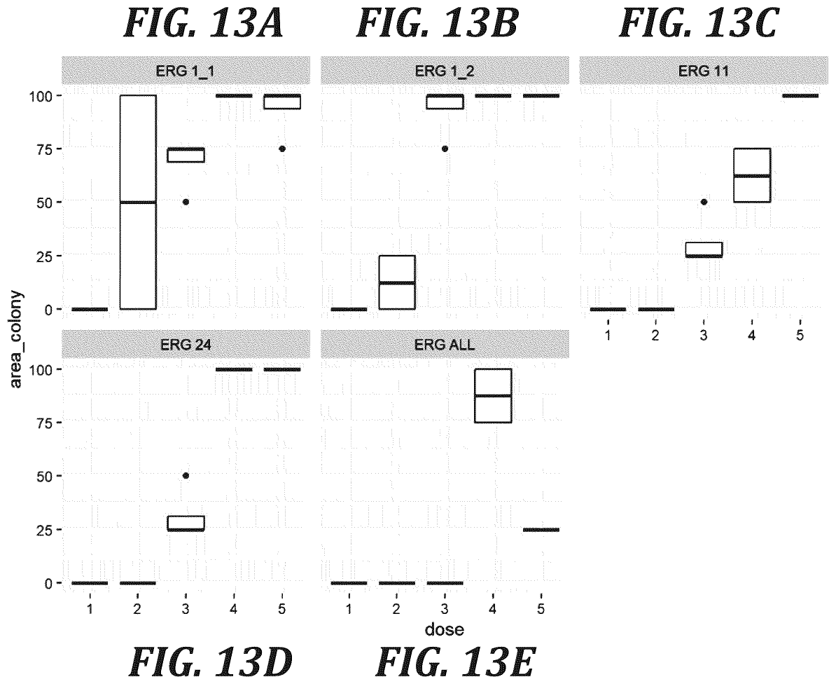

When colonies were treated 7 days after Pd inoculation using targets ERG1_1, ERG1_2, ERG11, and ERG24, the colonies treated with 5 doses exhibited 100% growth inhibition. Target ERG_ALL was not as effective regardless of the number of repeated doses (FIG. 13E).

Because many dose x treatment combinations resulted in the same response, reported below are (i) statistical results for the percent areas inhibited at the end of the study when pooling the results among the number of doses and (ii) the number of doses achieving total control or near total control. The results at the end of the study are visualized with box plots of all treatments, doses (1-5), and initial dose timings (Day 7, 21, 35).

### Initial Dose on Day 7

Pooling across all doses (1-5), target ERG1_1 applied 7 days after Pd inoculation controlled an estimated 62.5% of colony area (SE 11.46, p=2×10⁻⁷) with 100% control after 4 doses and near 100% control after 5 doses. Target ERG1_2 controlled 61.3% of the colony area (SE 11.46, p=4×10⁻⁷) with 100% control with 4 or more doses and near 100% control with 3 doses. Target ERG11 controlled only 39% of the growth (SE 1.14, p=0.0009), but 100% control was observed for 5 doses. Target ERG24 controlled 46% of the growth (SE 11.46, p=9.08×10⁻⁵) and 100% control when at least 4 doses were administered (FIGS. 13A-13E).

### Initial Dose on Day 21

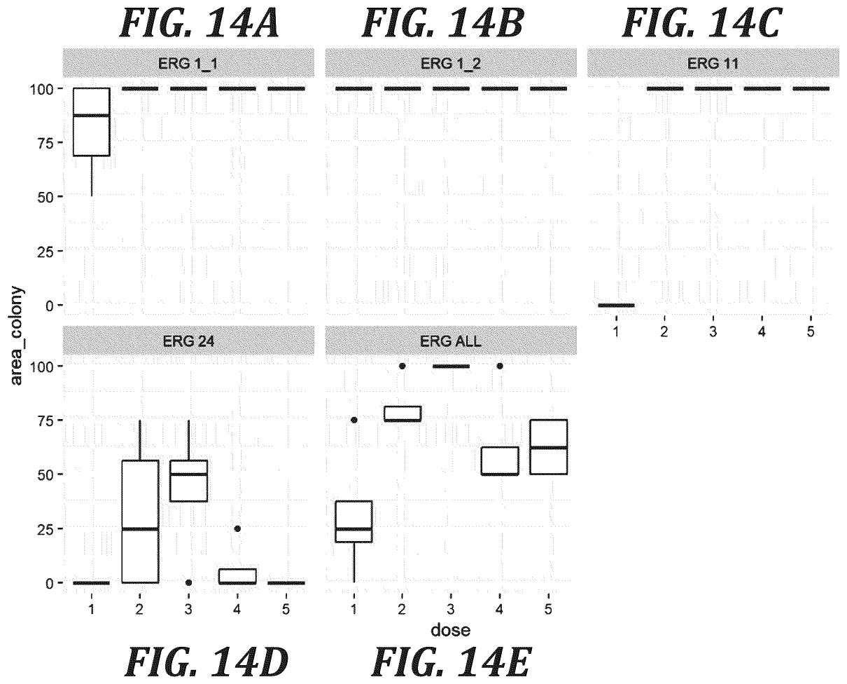

Colonies initially treated after 21 days had the strongest response. Two or more doses with targets ERG1_1, ERG1_2 and ERG11 experienced total inhibition of fungal growth (FIGS. 14A-14E). Up to 75% of fungal growth was controlled after two and three applications of ERG24. However, additional doses did not have the same effect as fungal regrowth was observed. The combination of all constructs, ERG_ALL, had up to 100% effectiveness after three doses and 50-75% after four or five doses. Limited effectiveness of ERG_ALL can potentially be explained because the most effective dsRNA treatments were diluted to achieve the same total volume of dsRNA.

Pooling across all doses (1-5), target ERG1_1 applied 21 days after Pd inoculation controlled an estimated 96% of colony area (SE 7, p<2×10⁻¹⁶). Target ERG1_2 uniformly controlled 100% of the colony area for all dose numbers. Target ERG11 controlled only 80% of the growth (SE 7, p<2×10⁻¹⁶). Target ERG24 controlled only 16% of the growth (SE 7, p=0.02). ERG_ALL controlled 68% of growth (SE 6.8, p=p<2×10⁻¹⁶) (FIGS. 14A-14E).

### Initial Dose on Day 35

The results when the initial dose occurred after 35 days of growth were more variable, likely because fungal colonies were too large to be fully treated with only 20 μL of our dsRNA suspension. Nevertheless, ERG1_1 remained highly effective, controlling 100% of fungal growth for 2, 3, and 5 doses and nearly 100% for 4 doses.

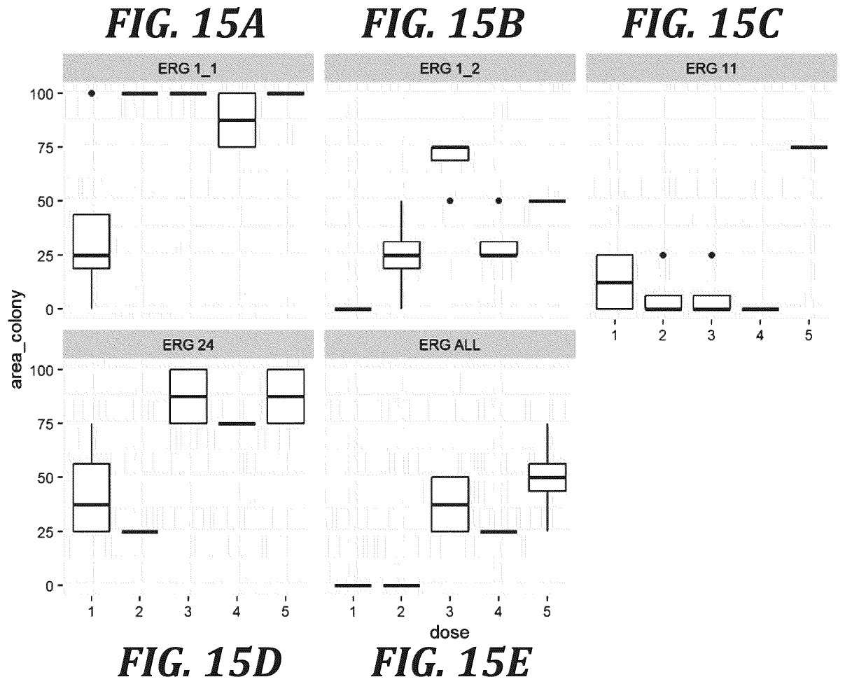

Pooling across all doses (1-5), target ERG1_1 applied 35 days after Pd inoculation controlled an estimated 85% of colony area (SE 7.4, p<2×10⁻¹⁶). Target ERG1_2 controlled 35% of the colony area (SE 7.4. p=6×10⁻⁶). Target ERG11 controlled only 20% of the growth (SE 7.4, p=0.008). Target ERG24 controlled 64% of the growth (SE 7.4, p=2×10⁻¹⁴). ERG_ALL controlled 23% of growth (SE 7.4, p=0.003) (FIGS. 15A-15E).

The amount of ergosterol using HPLC in control samples was higher than the amount of ergosterol in the treated colonies (Table 3).

TABLE 3
Ergosterol content in control colonies and colonies
collected after 4 and 5 doses of dsRNA treatment

| | | Content | Content |
|---|---|---|---|
| ID | Sample | (ug/ml) | (ug/g) |
| 1 | ERG24 control | 28.414 | 66.141 |
| 2 | ERG11 control | 24.399 | 60.038 |
| 3 | ERG11 (4 doses) | 12.846 | 27.426 |
| 4 | ERG1_2 (5 doses) | 15.26 | 28.641 |

### Viability After Repeated dsRNA Applications

Pd viability was evaluated by subsampling and plating treated colonies after the post-germination experiment was concluded. 5 μl per well from 96-well plates was applied to SDA petri dishes and fungal colony growth was evaluated through time. All petri dishes were incubated at 7° C. and colonies were monitored weekly for 90 days.

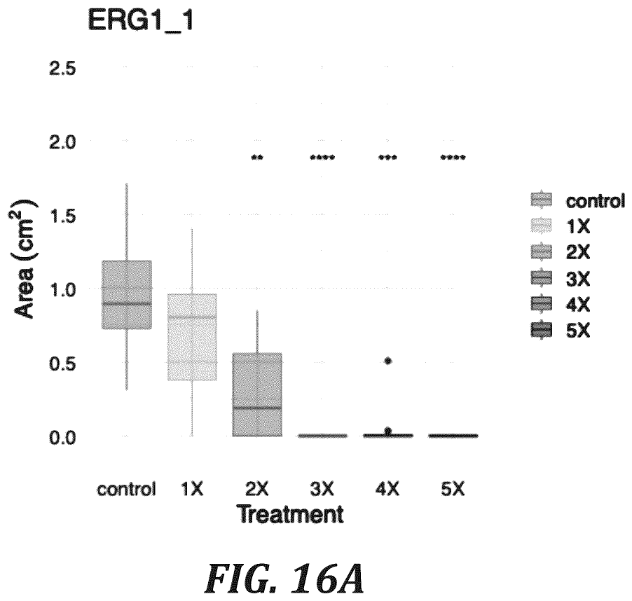

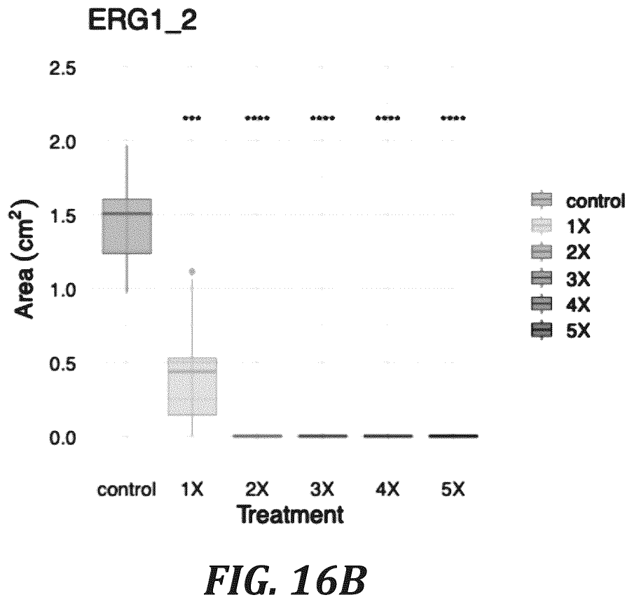

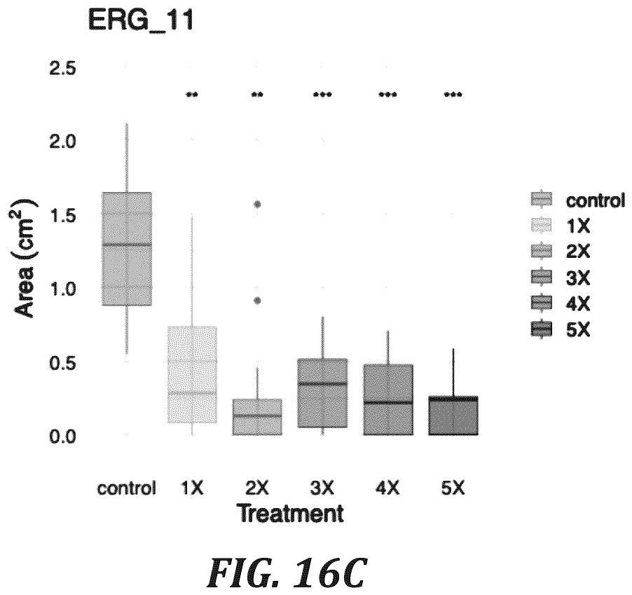

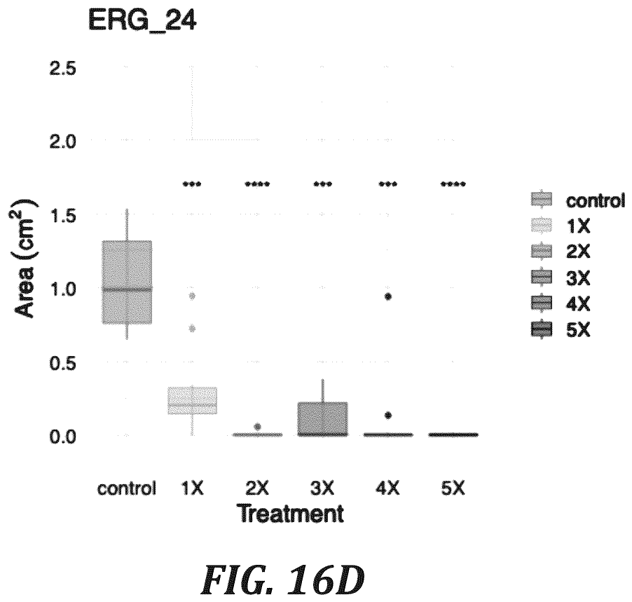

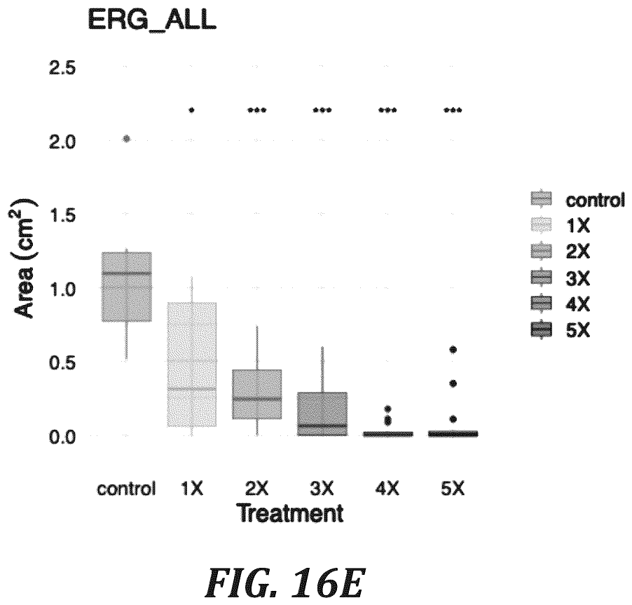

FIGS. 16A-16E compares Pd viability after repeated dsRNA applications. To determine if ERG treatments rendered Pd inviable, Pd growth was monitored for 60 days after 1, 2, 3, 4, or 5 repeat treatments, with the first treatment applied on day 7, 14, or 28 after fungal plating. 5 μl of the treated colonies was then replated. If growth was not seen in replated colonies after 90 days, it was concluded that a dsRNA treatment had rendered Pd inviable. Growth inhibition was observed in all replated colonies. Three dsRNA treatments (ERG1_1, ERG1_2, and ERG_24) rendered Pd inviable. Colonies treated with ERG1_2 dsRNA were rendered inviable after two doses. Reductions in colony regrowth were statistically significant versus the control in all treatments (p<0.05), apart from colonies treated once with ERG1_1 before harvesting.

### Repeated Dose Experiment for Chitin Synthase

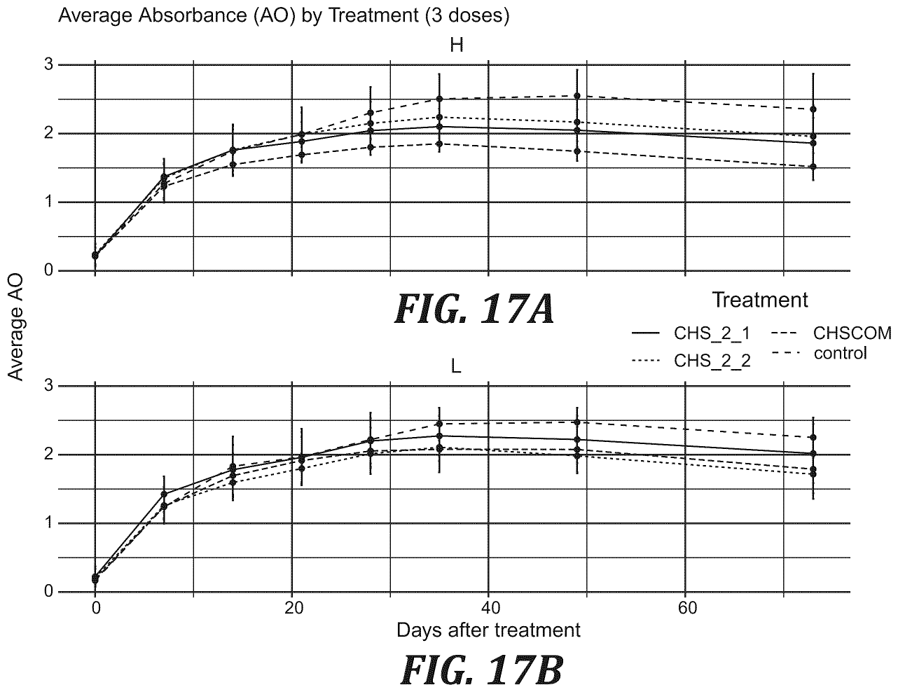

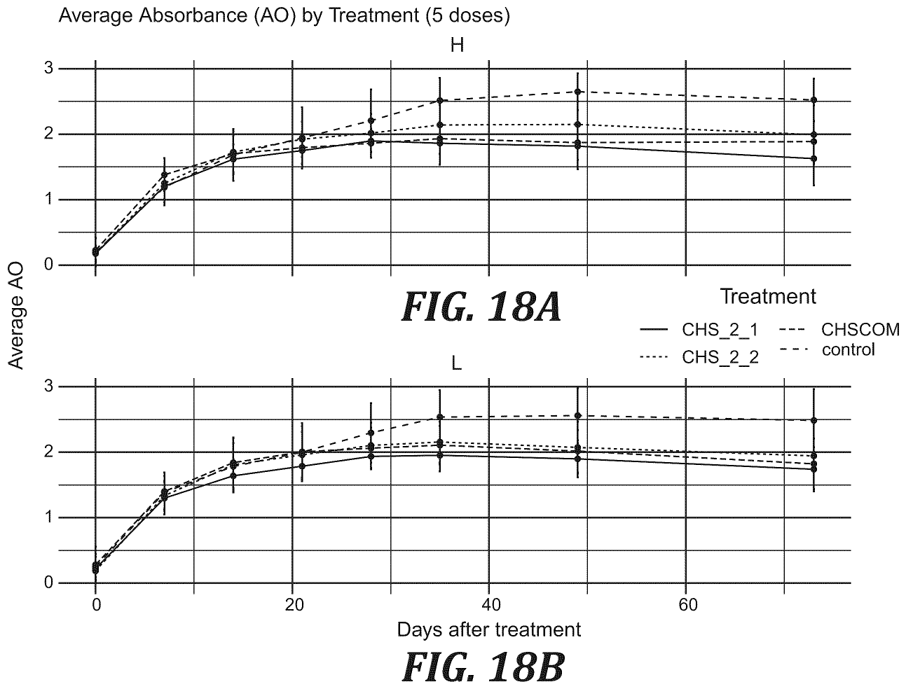

To determine if an RNAi control targeting the chitin biosynthesis (CHS) pathway effectively inhibited Pd growth, weekly applications of different CHS dsRNA treatments were applied in 96 well plates. High (5 μg/μL) and low (1 μg/μL) concentrations of three treatments (CHS2_1, CHS2_2) and a combination of both targets with the same concentration per target (CHS_combo) were evaluated using three (FIGS. 17A and 17B) and five applications (FIGS. 18A and 18B).

The treatments were assigned as follows per each type of application:

Columns 1-3 (wells A-G): Inoculated with Pd alone.

Column 4 (wells A-G) & Column 5 (wells A-D): Inoculated with Pd+CHS2-1 (high concentration).

Column 5 (wells E-H) & Column 6 (all wells): Inoculated with Pd+CHS2-1 (low concentration).

Column 7 (wells A-G) & Column 8 (wells A-D): Inoculated with Pd+CHS2-2 (high concentration).

Column 8 (wells E-H) & Column 9 (all wells): Inoculated with CHS2-2 (low concentration).

Column 10 (wells A-G) & Column 11 (wells A-D): Inoculated with CHS_combo (high concentration).

Column 11 (wells E-H) & Column 12 (all wells): Inoculated with CHS_combo (low concentration).

Column 1 to 3 wells A-G were inoculated with Pd alone, column 4 wells A-G and column 5 wells A-D were inoculated with a combination of Pd and CHS2-1 in the high concentration. Column 5 wells E-H and all wells in column 6 were inoculated with the low concentration for the same target. Column 7 wells A-G and column 8 wells A-D were inoculated with CHS2_2 in the high concentration. Column 8 wells E-H and all wells from column 9 were inoculated with CHS2_2 in the low concentration. All wells in column 10 and wells A-D in column 11 were inoculated with the CHS_combo in high concentration and the wells E-H in column 11 and all wells in column 12 were inoculated with CHS_combo in low concentrations. Absorbance measurements were collected weekly until (through) seven days following the fifth treatment dose. Additional absorbance measurements were taken two months after and again near three months after the experiment's initiation to monitor Pd growth over time.

### Repeated Doses Experiment for Chitin Synthase results

All control wells, which were treated weekly with water weekly, showed no inhibition on fungal growth and reached a plateau in its growth represented by the absorbance measure around day 50. In contrast, Pd growth was inhibited by dsRNA following three or five applications. The extent of inhibition varied depending on the target gene and dsRNA concentrations.

The most effective treatment following five applications is CHS2_1, which resulted on (in) a reduction of Pd growth. In both high and low concentrations, Pd reaches a plateau around day 28 post-initial treatment with CHS2_1, compared to controls that start reaching a plateau approximately seven days after. With only three applications, the most effective response was observed with CHS_combo in the high concentration. Notably, all dsRNA treatments led to some degree of growth inhibition, as Pd exhibited a reduction in growth after being treated with dsRNA.

The inhibition through time for all CHS targets, number of doses and concentrations are summarized in FIGS. 17A (high concentration) and 17B (low concentration) for absorbance after 3 doses and FIGS. 18A (high concentration) and 18B (low concentration) for absorbance after 5 doses.

The following examples are provided for the purpose of illustrating the invention.

### EXAMPLES

### Example 1

### Identification of Ergosterol Gene Targets in Pd

The UniProt database (Uniprot Consortium 2023) was queried for ergosterol biosynthesis genes within Pseudogymnoascus destructans (Pd), which produced hits for ERG1_1, ERG1_2, ERG11, and ERG24 proteins. tBLASTn (Camacho et al. 2008) queries of the amino acid sequences for these proteins on GenBank were performed to locate nucleotide sequences associated with these genes. The structure of these proteins associated with ergosterol biosynthesis genes using Phyre2 (Kelley et al. 2015) was verified.

Separate, custom target and nontarget databases were created in the si-Fi (Lück et al. 2019) siRNA finder tool using entire genomes from (1) Pseudogymnoascus destructans and (2) nontarget fungal species, including Acremonium strictum, Alternaria alternata, Aspergillus niger, Aspergillus versicolor, Aureobasidium pullulans, Beauveria bassiana, Fusarium solani, Penicillium chermesinum, Pseudogymnoascus roseus, Pseudogymnoascus verrucosus, and additional Pseudogymnoascus spp., and nontarget vertebrate species, including Artibeus jamaicensis, Carollia brevicauda, Cynopterus sphinx, Desmodus rotundus, Eptesicus fuscus, Hypsignathus monstrosus, Macrotus californicus, Miniopterus schreibersii, Murina leucogaster, Myotis brandtii, Myotis davidii, Myotis lucifugus, Myotis ricketti, Pteropus alecto, Pteropus vampyrus, Rhinolophus ferrumequinum, Rousettus aegyptiacus, Tadarida brasiliensis, Carollia perspicillata, Homo sapiens, Sorex araneus, and Sus scrofa.

si-Fi (Lück et al. 2019) was used to identify regions between 200 and 300 nucleotides in length that maximized the number of short-interfering RNAs (siRNA) in Pd and avoided off-target interactions.

### Example 2

### Identification of Chitin Synthase Gene Targets in Pd

UniProt database (Uniprot Consortium 2023) was queried for or chitin synthase genes within Pseudogymnoascus destructans, which produced hits for CHS2_1, CHS2_2, and CHS3 proteins. tBLASTn (Camacho et al. 2008) queries were performed of the amino acid sequences for these proteins on GenBank to locate nucleotide sequences associated with these genes. The structure of these proteins was associated with chitin synthase genes using Phyre2 (Kelley et al. 2015) were verified.

Separate, custom target and nontarget databases in the si-Fi (Lück et al. 2019) siRNA finder tool were created using entire genomes from (1) Pseudogymnoascus destructans and (2) nontarget fungal species, including Acremonium strictum, Alternaria alternata, Aspergillus niger, Aspergillus versicolor, Aureobasidium pullulans, Beauveria bassiana, Fusarium solani, Penicillium chermesinum, Pseudogymnoascus roseus, Pseudogymnoascus verrucosus, and additional Pseudogymnoascus spp., and nontarget vertebrate species, including Artibeus jamaicensis, Carollia brevicauda, Cynopterus sphinx, Desmodus rotundus, Eptesicus fuscus, Hypsignathus monstrosus, Macrotus californicus, Miniopterus schreibersii, Murina leucogaster, Myotis brandtii, Myotis davidii, Myotis lucifugus, Myotis ricketti, Pteropus alecto, Pteropus vampyrus, Rhinolophus ferrumequinum, Rousettus aegyptiacus, Tadarida brasiliensis, Carollia perspicillata, Homo sapiens, Sorex araneus, and Sus scrofa.

si-Fi (Lück et al. 2019) was used to identify regions between 200 and 300 nucleotides in length that maximized the number of short-interfering RNAs (siRNA) in Pd and avoided off-target interactions.

### Example 3

### Validation of Efficacy of dsRNA Treatments on Pd Colonies

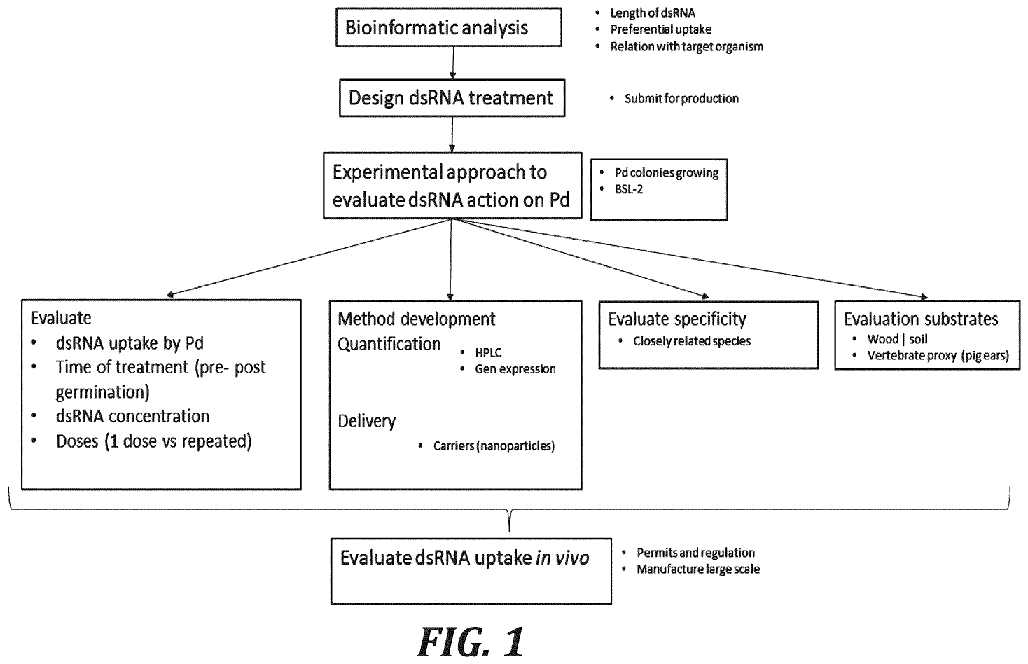

To validate the efficacy of multiple dsRNA treatments on Pd, different steps were used as part of the workflow (see FIG. 1).

### Sequences

Sequences Associated with Ergosterol Biosynthesis

ERG1_1 gene (1683 bp)
(SEQ ID NO: 1)
ATGATAATCTCTGAAGTGATTCCCTGCTGCCGTCTCCTTCCGCCGTTCCAC
CACATAAAGTACATAAACACCCTCGTCCAGCTGCGCAGCTTCAACAGCCCGCCC
ACCACCGCCCAAGTCAGCGTCCCAGCCAAAATTGCGTCGCCTGTCTCGCGCCCCT
TCACCTCCCTCCTCGCTCCCACAATCGACACCCAGTACCCCTCCATGACGGCGAC
GCAGACCACGCACGAGTCACTCTCGCAGCGGCGCCGAATGCACCACGAAGCCGA
TGTTGTTATAATCGGGGGGGGCGTCTTCGGGTGCGCCGTGGCGGTCACGTTGGCG
CGCCAGGGCCGCAGCGTCATTCTCCTCGAGCGGTCGATGAAGGAGCCTGACCGG
ATCGTGGGGGAGCTGCTGCAACCCGGCGGCGTGTCGGCATTACACAAGCTGGGA
TTGTCGGAGTGTCTGGAGGGCATTGATGCGGTGGTTGTTAATGGCTATAATGTCA
TCTACTACGGGAAGGAGGTTCATATCCCGTACCCGTACGATACGAGGGTGGAGA
AGTCGAGGGCGATGAGGGAGTCAAGGCCGGTGGGGTGGTGTTTTCACCACGGAC
GGTTCATTAACAAGCTCCGCGAGGCGTGCAAGAGCGAGCCGAATATCACGATCT
TCGAGACGACGGTGAAGGGGACGGTGAGCACGGATAACAACGAGCAAGTCCTC
GGCGTAAAGACAGAGACGACAGACCCCATGGACGGGTCGAAGAAACCGGATTA
CTTCTTCGGCGGCCTCACCATCGCAGCAGACGGCTACGCATCCACCCTGCGAAA
ACAGTACATCTCCAAGACCCCCGTGGCGAAGTCCAAATTCTACGCCCTCGAACT
CATAGACTGCCCCATCCCAACCCCGAATCACGGCCACATCATCCTCTCCGACAAT
GCCCCCGTCCTCGTCTACCAGATCGGCACCCACGAGACCCGCGCGCTCATCGAC
GTCCCCGAGAACCTGGAGACCGCCAAAGCCGCCCTGGGGGGCGTAAAAGCACA
TATTCGCAATGTCGTCGTGCCAACGCTTCCGAAGCAAATCAAGCCGACTTTTATA
AGGGCGCTGGAGGAGGGGAAACTAAGGAGTATGCCGAATTCGTGGTTGCCGCC
GACGCAGCAGCAGACCCCGGGGATTGTGGTACTGGGGGATGCGATGAACATGC
GTCACCCCCTCACCGGCGGCGGCATGTCCATCGCCCTCACCGACGTGGTCATCCT
CTCCGAGCTCCTCCACCCGACTCGCATCGCGGACCTCTCCTCCGTCCCCGCCATG
AGCCTCGCAATGCGCACCTTCCACTGGCGGAGGAAACAACTCGCATCCATCGTG
AACATCCTCGCCCAAGCCCTCTACGCCCTCTTCGCCGCCAACAACGCCGAGCTGA
AAGCCCTGCAGCGGGGGTGCTTTGAGTACTTCCTCTTTGGGGGCGCGTGCATTGA
TGAGCCGGCGGGGATGCTGGCATGTATCCTGCCGAGGCCGTTTTTGCTGTTCTAC
CACTTTTTCTCGGTGGCGCTGCTAGCGATGTGGTTGATTATGTGTGATTGTGTGG
GGGATTTGCTGGGGGTGTGGAGGGCGCCGCTGGGGGTGTATAGGAGTGGTGCGG
TGTTGTGGAAGGCGGTGGGGGTGATTTTTCCGTATATTTTTTCCGAGTTGACGTG
GTGA
ERG1_1 dsRNA Target (258 bp)
(SEQ ID NO: 2)
CTGCTGCCGTCTCCTTCCGCCGTTCCACCACATAAAGTACATAAACACCCT
CGTCCAGCTGCGCAGCTTCAACAGCCCGCCCACCACCGCCCAAGTCAGCGTCCC
AGCCAAAATTGCGTCGCCTGTCTCGCGCCCCTTCACCTCCCTCCTCGCTCCCACA
ATCGACACCCAGTACCCCTCCATGACGGCGACGCAGACCACGCACGAGTCACTC
TCGCAGCGGCGCCGAATGCACCACGAAGCCGATGTTGTTATAAT
ERG1_2 Gene (1566 bp)
(SEQ ID NO: 3)
ATGACTTTCGGTACTACCTCACAGAATCTCTACAACGCTCAAAGACGCTC
CGAGCACCATGAGGCCGATGTCCTGGTGGTCGGCGCTGGAGTGTTTGGCTGCGC
GATGGCTTATGCGTTAGCTAACCAAGGGAGGAGTGTTATTCTGCTGGAGCGGCA
GATGAAAGAGCCGGACCGCATTGTCGGGGAGCTTCTCCAACCCGGTGGTGTGGA
GGCGTTGAAGAAGCTTGGGCTAGAGAAATGCCTGGAAGGTATTGATGCCATTCC
TTGCTACGGCTACAACGTCCACTACCACGACGACGACGTCGTGATCCCGTACCCT
GCCATCGAGCCTTCGGGTAAGGTCTTAGCGGCCACTCACAGAGGCCTTACTGCC
CAAGAGTACTTGCAGGCGGTTGGTAGGAACAATACAAATGGCCATGCAGATAAA
GCTAGTGGAGCCGTGCACAAGTTCTCTCTCGACAAGCCCGAGGGCCGTAGTTTTC
ACCACGGACGGTTTATTATGCGGCTGCGACAATCCTGCGACAATCACCCAAACG
TCACTATTTTCGAGACAGAAGTGACGAACACGATCCGGTGCGAGCACACCTCTC
AGATCCTCGGTGTGGAGACGAAGAGAGCGGACAAAACAAAGGATTACTTTTTTG
GCCAGCTCACTGTCATCGCCGACGGGTACAATTCCAGGTTCCGCAAGCAGATCA
TCAACAAGACTCCCATTGTTAAGAGCAAGTTTTACGCCTTGACGCTTATCGACTG
CCCCATGAAGCCTACGAATTTTGGCCACGTCATCATTGGCAAAGCTTTCCCCATT
CTAATGTACCAGATTGGCAACCACGAGACGCGCGCGCTAATCGACGTGCCCGAG
AACCTCCCGGAGGCATCCCCTCAGAACGGTGGCGTTCGTGGCTATATCAGAAAC
GTCGTGATTCCAACTCTTCCAGAAGGAACAAAGCGGGCATTTGAGTTGGCCCTG
GCTAGTGGCCGTATCCCGAAGAGCATGCCAAACAGCTGGCTCCCACCGGTGCGC
CAGAAGAACAACTTGGGCGTCGTCCTCCTCGGCGACGCTTACAATATGCGTCAC
CCACTAACAGGCGGCGGCATGACTGTAGCCTTCAATGATGTCGTCCTCCTCTCGA
AATTACTCCATCCGGACAAGGTCGCTGACCTCGGCAACACCGACGCCATTAACG
CGGCCATGAGCAAGTTCCACTGGCAGCGCAAGTCGCTCACCAGTATTATTAACG
TGCTTGCCATGGCGCTTTATATGCTCTTTGCAGCACAGGACCGCCAGCTTGCTGC
TTTGCAGCGCGGCTGCTTCGTCTACTTCAAAAAGGGCGTTACTGATGAGCCCAGC
GCCATGCTAGGCGGTATCCTCCACCGGCCCTCTGTGCTGGCATACCACTTCTTTG
CCGTAGCTTTCCTATCTATCAGGATCAACACCGTCGACCTTTGCGGGGGGGGCAT
CTCCGGCATTTGCAAGGTACCCCGAGCCGTTCTCGACGCTGTGCTGATCCTCTGG
AAGGCGTCTGTTGTCTTTCTGCCCGTCATGTGGCAGGAACTGCAGTAA
ERG1_2 dsRNA Target (200 bp)
(SEQ ID NO: 4)
AGCTCACTGTCATCGCCGACGGGTACAATTCCAGGTTCCGCAAGCAGATC
ATCAACAAGACTCCCATTGTTAAGAGCAAGTTTTACGCCTTGACGCTTATCGACT
GCCCCATGAAGCCTACGAATTTTGGCCACGTCATCATTGGCAAAGCTTTCCCCAT
TCTAATGTACCAGATTGGCAACCACGAGACGCGCGCGCTA
ERG11 Gene (1590 bp)
(SEQ ID NO: 5)
ATGGGCCTAACAGAGAGCCTTACTGAGGCCGTCCTGATCCCAATCAATGC
GCAGATTTCGGAGCGTGGTCTCGGCGTCGTGGCAGCCGTCGGATTCGCGTCGTTC
ATCGTCCTCGCTGTCGTCCTCAATGTCTTGAGCCAGCTTCTTCCCCAAAACCCCA
ATGAGCCGCCGCTTGTTTTCCACTGGTTCCCCATCCTCGGAAACACTATCAGCTA
TGGAATGGACCCATACCCCTTCTTCTTCAAGTGCAGAGAAAAGTATGGTGATATC
TTTACTTTTATCCTCCTCGGAAAGAAGACCACCGTATACCTTGGAACAGCTGGCA
ACGAGTTCATCCTCAACGGCAAGCTCAAGGACGTGAACGCGGAGGAGATCTACA
CAAAGCTCACCACGCCAGTTTTCGGTACCGGAGTTGTCTACGACTGCCCCAACTC
GAAGCTCATGGAGCAGAAGAAATTCGTGAAAGTCAGTCTCACCACCGAGACATT
CAGGTCCTACGTGCCCCTTATCGTCCAGGAGGTCACCAACTTCATCAAGACCAGC
CCGATGTTCAAGGGTCCCAGCGGTAAAACCGACGTTCCCGCAGCCATGTCTGAG
ATCACCATCTACACCGCTTCCCGTACACTCCAAGGGAAGGAAGTCCGATCCAGG
TTCAACGCCGAATTTGCCAACCTCTTCCACGACCTCGACATGGGCTTCACCCCGA
TCAACTTCATGCTTCACTGGGCACCTCTGCCACAAAACCGTGCTCGCGATCGCGC
TCAGAGAATCCTTTCCGAGACATACATGGAGATCATCCAGGAACGTCGCGCCGG
AAACGTCCCTGATGAGAATGAGCACGATTTGATCCGACATCTGATGGCATCGGT
TTACAAGGACGGCACCCCTCTCCCTGACAAGGAGATTTCACACATGATGATTGC
CATGCTCATGGCGGGACAGCACTCCTCGTCATCGACCAGCTCCTGGATGATGCTT
AACCTCGCCGCCCGCCCCGATATAATCGAGGGACTCTACCAAGAACAGATCCGC
CTCCTAGGCGCCGACCTCCCCCCCTTGACCTATGAGAACTTGGGCAAGCTCACCC
TGAACAACGCCGTCCTCAAGGAGACCCTCCGTCTCCACACCCCCATCCACTCCAT
CCTCCGCAAAGTCAAGTCCCCCATGCCCGTCCCCGGAACCAACTTCGTCATCCCA
ACAACCCACACCCTCCTCTCCTCCGCCGGTGTCACCGCGCGGATGGAGGAGTAC
TTCCCCGACCCTCTCAAGTGGGACCCCCATCGCTGGGACGCAGGCGCAGCCGGC
GTCCAGATGGGCGAGATCAAGGAGGAGCACGAGGACTACGGCTACGGCATGAT
CAGCAAGGGCGCCACGTCGCCCTACCTGCCCTTTGGCGCTGGCCGCCACCGCTG
CATCGGCGAGCAGTTCGCCGGCCTGCAGCTCGGGGCCATCATCGCGACCATGGT
GCGCGAGTTCAAGTGGAGGCTGCCTGACGGCGTGAACGAGGTTGTCGGCACGGA
TTACAGCTCGCTGTTCTCGCGGCCGCTGACCCCGGCCATGATTGTGTGGGAGAAG
CGGGAGAAGAACTAA
ERG11 dsRNA Target (219 bp)
(SEQ ID NO: 6)
ATCGAGGGACTCTACCAAGAACAGATCCGCCTCCTAGGCGCCGACCTCCC
CCCCTTGACCTATGAGAACTTGGGCAAGCTCACCCTGAACAACGCCGTCCTCAA
GGAGACCCTCCGTCTCCACACCCCCATCCACTCCATCCTCCGCAAAGTCAAGTCC
CCCATGCCCGTCCCCGGAACCAACTTCGTCATCCCAACAACCCACACCCTCCTCT
CCTCC
ERG24 Gene (1464 bp)
(SEQ ID NO: 7)
ATGGCCGGTTCGAAATCTAAAGCCCCCGCCGTGAAGGCGCAACAGAAAC
ATGGATATGAGTTCGCTGGACCGCCCGGTGCATTCGCCATCTCGTTCCTCCTTCC
GATCGTCGTCTACATCACCAATTTCGTCTGCAACGACATTTACGGATGCCCGATC
CCCTCGGTGCTGGACCCCAAGACGTTGACATGGGACAAGATCAAGACTGAGACT
GGATGGCCAGGATGGAATGGCATTGTGAGCTTCGAGGCTACTGGCTGGGTGTTG
GCATACTATTTCCTGAGTTTGGTGCTGCACAGATTCCTTCCGGGACAAATCGTTG
AAGGAACTGAACTGGCTATTGGAGGACGCTTGAAGTATAAGTTCAATACTCTTTC
CTCCTCCATCTTTACCGTCGTTCTCCTCCTCGCCGGTACTATCGCGCAGGGCGCC
GACTTCCCAGTCTGGACCTACATCTGGGACAACTACACCCAGATCGTCACCGCC
AACATGCTCATCGCCTTCACCCTCGCAACATTCGTCTACATCCGCAGCTTCAGCG
TCAAGCCCGGCAACAAAGACATGCGCGAGCTCGCCGCCGGTGGCCACTCCGGCA
ACATGCTCTACGACTGGTTCATCGGCCGCGAACTCAACCCGCGCGTCACCCTCCC
CATCTTCGGCGAGATCGACATCAAAGTCTTCTGCGAGCTGCGCCCAGGTCTCTTT
GGCTGGATCCTCCTCGATGTCGCCTTCATCGCGCACCAATACAAGACATACGGCC
ACGTCACCGACTCCATCATCCTCGTCACCCTCTTCCAGGCCCTCTACGTCTTCGA
CTCCTTCTACATGGAACCCTCCATGCTGACCACCATGGACATCACCACCGACGGC
TTCGGGTTCATGCTCTCCTTCGGTGACCTCGCTTGGGTCCCCTTCATCTACAGTCT
GCAAGCGCGCTACCTCGCCGTCTACCCCCTCACCCTCGGCCTCTCCGGCAACGCC
GGCGTCCTCGCCATCCTCAGTCTGGGGTACTACATATTCCGGAGCGCCAACAACC
AAAAGAACCGCTTCCGCACCGATCCGTCTGACCCGCGCATCGCGCATCTCAAGT
ACATGGAGACGGCCTCGGGGTCAAAACTCATCATCTCCGGATGGTGGGGGACGG
CGCGCCACATCAACTACCTTGGCGACTGGATCATGTCGTGGGCGTTCTGTCTGCC
TACCGGGATAGCGGGGTACCTTGTGCAGCAGGGCCCGGCGCTGTTGGATGGCGA
GACGGGATTCTCGCGCGGGGAGGATAGGGTGGTGCAGGGGGGTGCGAGGGGGT
GGGGAATGGTGATTTCGTATTTTTACGTTATTTACTTTGCGATTCTGTTGATTCAT
CGCGAGAGGAGAGATGAGACGAAGTGTAAGAGGAAGTATGGGAAGGATTGGGA
GGAGTATAAGAAGATTGTGCCGTGGAGGATTATTCCAGGGATTTATTAG
ERG24 dsRNA Target (245 bp)
(SEQ ID NO: 8)
GTTGAAGGAACTGAACTGGCTATTGGAGGACGCTTGAAGTATAAGTTCAA
TACTCTTTCCTCCTCCATCTTTACCGTCGTTCTCCTCCTCGCCGGTACTATCGCGC
AGGGCGCCGACTTCCCAGTCTGGACCTACATCTGGGACAACTACACCCAGATCG
TCACCGCCAACATGCTCATCGCCTTCACCCTCGCAACATTCGTCTACATCCGCAG
CTTCAGCGTCAAGCCCGGCAACAAAGACAT

Sequences Associated with Chitin Synthase

CHS2_1 Gene (3360 bp)
(SEQ ID NO: 9)
ATGTATCGCCCAAATACACCTCCAAACTTCGATCCTCCATCATACGGTGA
TGATGAGGGATCAACACCAACACACATGTCGAACGATGCGTCCGCCATCCGCCT
GTTGACATCAATGGACGACTCCAACTCCTCGAACGCCCGCATGTATGTACAGGA
TCCTGATTTACTACCCCGTCCTCTCGCGGTGCACAAGAAGTCTGTGCAGTTCTCC
ACGCTAGAACACATTTCTGCCCTTCATGGCGAAGAAGAAGCTATCTCGAGCGGC
AAGTTGCCAGCTGCGGTGACCAAGCAAGCGCCGCCGAAGCCCCCCCAAGAAGA
CCACTTCGTGAATCTCCTCCCCAAACTCCCTGATGGACCCTCGCGCAGGAAGTCG
CTGCGGAGTCAAGTCAAAGCCCACTTGGCTGCTAACGGTGAGATGAAGAATGAA
AGCTTACCTGTCAATAGTCCCAGCACCCCGCGCAGATCATACCAGCCTACCGTCA
TGTCGACGAGCTCGCGCTCCAATTCCATCGTTGATGGAGCGCCGATATTACCACC
GGAGAGCTCATACGACCCATACACCGGGAGACAGTCACCGACGCGGTCGTGGAC
CCCATCTCAAACAGCGTCTGAGAGAGGTAGGCCACCATCGATAGCTCAATATGA
GCCAGCGGATGTGAATGGCTCTCCGAGACCCGGCACTCCTTCGTCGAGATACGG
CGGCAGCCCGAGGAGACCACTCCCTCCCGCACCACTATTCTCTGGACCGGGTGC
TGGTGCGCGGAGCTCCACATTCGCAGACGACGCGACGGTTTCGATCGCGTTAGA
AACAGAAGGAGACGACGTTTTTGCTCCGAAAAGTACTATCGGTACTGATCATGT
CCGGTCCCAGTCTCGCGAGTCCTACACGTCAGAGTCGACGTTCACCGAGGAGTA
CGACAATGAGAAGTCCGACTTTGAGCACTACGGGCCTGCGCCAGATGGGCGACA
GGAGCGCAGAGGTGCTCGCCAGGCACAGATGGCCAAGAAGGAGGTTCGTCTCAT
TAACGGCGAGCTTATCCTCGAATGCAAGATCCCCACCATCTTGTACAGTTTCCTC
CCCCGCCGCGATGAGGTTGAGTTCACGCACATGAGATACACAGCCGTGACCTGC
GATCCCGATGACTTCGTGTCCAAGGGCTACAAGCTGCGCCAGAACATTGGCTCG
ACGGCCAGGGAAACAGAGCTCTTCATCTGTATCACCATGTATAACGAGGACGAG
ATCAACTTCACGCGCACCATGCACGCCGTCATGAAGAATATTGCCCATTTCTGCT
CGCGCTCCAAGTCGCGCACGTGGGGCGAGAACGGGTGGCAGAAGATTGTCGTCT
GCATCGTCTCTGATGGTCGGCAGAAGATCCACCCGCGCACGCTCGATGCGCTCG
CGGCGATGGGTGTGTACCAGGACGGCATTGCCAAGAATCTTGTCAACCAGAAGG
AGGTGCAGGCGCACGTGTACGAGTATACCACTCAGGTGTCGCTGGACTCGGACC
TGAAGTTCAAGGGCGCGGAGAAGGGAATCGTGCCGGTGCAGATGCTGTTCTGTC
TGAAGGAGAAGAACGCGAAGAAGCTGAACTCTCATCGCTGGTTCTTCAATGCTT
TTGGTCGCACTCTGACGCCGAATATCTGCATCCTGCTTGATGTGGGTACGAAGCC
GACGGGGAATTCTCTTTACCATCTCTGGAAGGCCTTTGACACCGACTCCAACGTC
GCAGGTGCCTGTGGAGAGATCGTGGCGATGAAGGGGAAGGGCTGGCTTGGGTTG
TTGAACCCGCTGGTTGCGTCGCAGAATTTTGAGTACAAGATGTCTAATATCTTGG
ATAAGCCGCTCGAGTCGGTGTTTGGATACATTACTGTCCTTCCTGGTGCTCTCAG
TGCGTATCGTTACCACGCTCTGCAGAATGACCATACTGGCCATGGACCGCTAAGT
CAGTACTTCAAGGGTGAAACTCTGCACGGCCAGAATGCCGATGTCTTCACGGCC
AATATGTATCTTGCTGAAGATCGGATTCTGTGCTGGGAGCTTGTCGCGAAACGAG
ATGAGAGATGGGTGTTGAAATATGTCAAGAACTGCAAGGGAGAGACTGATGTGC
CTGATACCGTCCCCGAGTTTGTCTCCCAGCGTCGCCGTTGGCTCAATGGAGCTTT
CTTCGCTGCCGTCTACTCTCTGGTGCATTTCAAGCAGATCTGGAACACAGACCAC
ACCATCGCACGCAAGATCCTCCTCCATATTGAATTCGTGTACCAGCTGCTCAGTC
TGCTCTTCACGTTCTTCTCCCTGGCGAACTTCTACCTCACGTTCTACTTTGTTGCT
GGCTCGCTCGCTGATCCCAAGGTCGATCCGTTTGGCCACAGCATCGGGAAATAC
ATCTTCTATATCCTGAAATATGTCTGCGTGCTTCTCATCTGCACGCAATTCATCCT
CTCCATGGGCAACCGGCCACAGGGCGCGAAGAAGTTGTACCTTTCTAGTATGGT
CATCTACGCAATCATCATGGTATACACGACCTTTGCGACGATCTACATCGTCGTG
CGGCAGTTTACCAGCAACACCATAACTCTCGGAAACAACATCTTCACCAACGTC
GCCGTGTCCATCGCCTCGACGCTCGGGCTGTACTTCTTCTCCTCCTTCCTATACCT
CGACCCATGGCACATGTTCACCTCTGCGGCGCAGTACTTCGCCCTCCTGCCCAGC
TACATCTGCACCCTCCAGATCTACGCCTTCTGCAACACCCACGACATCTCCTGGG
GAACCAAGGGCGACAACACCGTTCGCACCGACCTCGGCACAGCCGTCTCCAAAC
ACAAGGGCTCCACCGTCGAGCTCGACATGCCCAGCGAGCAGCTCGACATCGACT
CCGGCTACGACGAGGCCTTGCGCAACCTCCGCGATCGCATCGAGGTTCCCGAGC
TCGGCCCATCCGAGGAGACCGCACAGGAGGACTACTACCGCGCTGTGCGCACGT
ACATGGTCGTCTCGTGGATGGTGGCGAATGCCATCCTGGCCATGGCCGTGTCGG
AGGCGTACCAGGGCAAGAACATCGGGAGCAACTCGTACCTCAAGTTCCTGCTTT
GGTCTGTGGCGGCGATCGCGCTGTTCCGCGCGGTGGGTAGCACGACGTTCCGCA
TCATTGAGCTGGTGGGTATGCTTATTGACGGGAAGGCGAAGTGGGAGAGCGGGA
GCTACAGGTGGGGGGGTAGCTCGGTGGGGGGGAGCACGGTGCTTAGCTCGAAG
GCTAGGGGGGGTGGGTTTTGGTCGAAGTTTGGGTTTGGGAGTGTCAAGGATAAG
ATTAGTGATATCTCGAGTAGTATCGGGAGCAGTGTTTCGAGGAAGTGA
CHS2_1 dsRNA Target (243 bp)
(SEQ ID NO: 10)
TCTCATCTGCACGCAATTCATCCTCTCCATGGGCAACCGGCCACAGGGCG
CGAAGAAGTTGTACCTTTCTAGTATGGTCATCTACGCAATCATCATGGTATACAC
GACCTTTGCGACGATCTACATCGTCGTGCGGCAGTTTACCAGCAACACCATAACT
CTCGGAAACAACATCTTCACCAACGTCGCCGTGTCCATCGCCTCGACGCTCGGGC
TGTACTTCTTCTCCTCCTTCCTATACCT
CHS2_2 Gene (2718 bp)
(SEQ ID NO: 11)
ATGGACGGTCGTTACAATCCAGGGGCATCTCTTCGTGAGGTGGATCCTTC
GAATCCTGGATATAACCAACCTAGGCCAGACACTCGCGATGAATCCGAACTGGG
CCTGCTATCCCCAAGCGGTGGGCATGCATACCAGTCCCCATTTGATGGCAACAAT
CAATCGAACCTGGAAATTCAGAGGCCTGTCTCGACAGCATACAGCTTGACAGAG
TCGTACGCGGTGCCTGGGCAGCAGAACCCACACCACGCAACAGAGTATTCGAGC
AGCTCATCCTTCCAGCAAGGAATCGAACTCGAGGACATTCCTTTCGGAGGCACA
AACCGAGCCCCGTCGCCATCGCGAACGATCGACTCGGAAGATGCCTGGAGAAAG
CGTCAGGCTCCTGGCGGCGGACTCAAGCGCTACCCTACAAGAAAGGTGAAGCTT
ATTCAGGGCTCGGTCCTCAGTATCGACTACCCAGTCCCGAGTGCCATCAAGAACT
CGATAGAGAAGAAATACACCGCCGATGTCGAAGCTGGGAATGAAGAATTCACC
CACATGCGATACACTGCGGCTACCTGCGACCCCAACGACTTCACAATGAGGAAC
GGCTACAATTTGAGGCCCGCGATGTACAATAGGCACACCGAGCTCCTGATTGCA
ATTACATACTATAACGAAGATAAGGCGCTGCTTTCGCGCACGCTGCACGGTGTC
ATGCAGAACATCAACGACATTGTCCGTCTCAAGAAGACTGAGTTCTGGAACAAG
GGAGGACAGGCATGGCAAAAGATTGTCGTCTGCTTGGTCTTCGATGGTTTTGATG
CTTGCGACAAGGACGTTCTTGACGTTCTGGCCACGGTCGGTGTCTTCCAAGAAGG
TGTCATGAAGAAGGATATCGACGGTAAGGAGACAGTTGCCCATATCTTCGAGTA
CACGACACAGATCTCGGTGACTGCCAACCAGCAGCTTGTGCGCCCTCAAGAGGG
TGCGGCAAACAACCTGCCACCCGTGCAGATGATGTTCTGCTTGAAGCAGAAGAA
CAGCAAGAAGATCAACTCCCATCGTTGGTTGTTCAACGCTTTCGGGCGTATTCTG
AACCCAGAGGTCTGTATTCTGCTGGATGCCGGTACTAAGCCTGGTCCCAAATCGC
TCTTGTCGCTGTGGGAAGGTTTCTTCAACGACAAAGATCTCGGAGGAGCTTGTGG
TGAAATCCACGCCATGTTGGGCAAGGGTGGGCGCAACCTGATCAACCCTCTGGT
TGCAGCCCAGAACTTCGAGTACAAGATCAGTAACATTCTCGACAAGCCGCTTGA
GAGTTCTTTCGGATATGTCAGTGTGTTGCCGGGTGCTTTCTCAGCATACCGTTAC
CGTGCTATCATTGGCCGCCCGCTCGAGCAGTACTTCCATGGTGATCACACGCTTT
CTGCGATTCTTGGAAAGAAGGGTATCGAGGGCATGAATATTTTCAAGAAGAACA
TGTTCTTGGCTGAGGATCGTATTCTTTGCTTCGAGTTGGTTGCCAAGGCTGGCTC
GAAGTGGCATTTGACATACATCAAGGCGGCAAAGGGAGAAACGGATGTTCCTGA
GGGCGCCCCTGAATTCATTGGCCAGCGTAGAAGATGGCTCAACGGTTCATTTGCT
GCCAGTGTCTACTCCGTTATGCACTTTGGTAGAATGTACAAGAGTGGACACAACT
TCGTCCGCATGTTCTTCTTCCATATCCAACTTCTTTACAACATCTTCACGGTTATC
CTCACTTGGTTCTCGCTCGCATCTTACTGGCTTACAACGACTGTCATTATGGATCT
TGTTGGTCTTCCAGAGCCAGCTTCGGGTACCGGAGCAGAGCATCACGGATGGCC
TTTCGGAGACACAGCGACGCCAATTATCAACACGATTCTGAAGTACCTATACCTC
GGATGTCTGGTCATGCAATTCGTGTTGGCCCTCGGAAATAGGCCAAAGGGATCC
CGCTACACTTACATCACATCGTTCATGATCTTCTCATTCATTCAGTIGTATGTCAT
TATTTTGTCTATGTTCTTGGTTGTACGCGCGTTTGTCGGACCAAACGTGGACAAA
ACAGCGATCTTCACCACCAATATCTTCTCGTCAAACAGCTCCGCTATCATTCTCA
TCGCGCTGGCGGCCACCTTCGGTTTATACTTTGTCGCCTCGTTCCTCTACCTGGAC
CCATGGCACATGTTCCACTCCTTCCCTCAATACCTGCTTGTTGCATCTTCGTACAT
CAACATCCTCAACGTCTACTCCTACAGCAACTGGCACGATGTGTCTTGGGGAACC
AAGGGATCGGACAAGGTCGATGCTTTGCCATCTGCACAGACAACCAAGGTTGAG
GGCAAGGCTGCTGTCATCGAAGAAGTCGACAAGCCACAAGAGGATATTGATAGC
CAGTTTGAGGCAACAGTCAAGCGCGCTCTCAAGGAATACAAGGCACCCAAGGA
GGATGAGTCCAAGTCTTTGGAGGATTCGTACAAGTCTTTCAGAACGACCCTGGT
GGCTTTCTGGATTTTCTCCAACGCCATTCTTGCGGTGGTCATTACTAGCGACAAC
TTCGATTCGTTCGGATTCGGGAACAAAGCATCTGCTCGCACAGCCAAGTTCTTCC
AGGCCCTGCTCATCGCTACTGTCTGTCTGTCTCTCGTTCGCTTTATTGGATGCCTG
TGGTTCCTGGGTAAGTCCGGAATCAAGCGCTGCTTCAGGAGACGTTGA
CHS2_2 dsRNA Target (250 bp)
(SEQ ID NO: 12)
TTCGTGTTGGCCCTCGGAAATAGGCCAAAGGGATCCCGCTACACTTACAT
CACATCGTTCATGATCTTCTCATTCATTCAGTTGTATGTCATTATTTTGTCTATGTT
CTTGGTTGTACGCGCGTTTGTCGGACCAAACGTGGACAAAACAGCGATCTTCAC
CACCAATATCTTCTCGTCAAACAGCTCCGCTATCATTCTCATCGCGCTGGCGGCC
ACCTTCGGTTTATACTTTGTCGCCTCGTTCCTCT
CHS3 Gene (3627 bp)
(SEQ ID NO: 13)
ATGTCCCTACCCCAGAGACCTGGGGGCGGCGACCTCCCCCCCCAAGAACG
GCGAAATACGTATAGAAGCTCCACTGGGCGCCCTCAACCGTCACACGACGCCGA
AGTTGGATATGCTGGGCAAGATTCGCAATCTCCAACATCTAGACGGACAAGACA
ACATCGGACAGAACGAGCCGGGTCTCCCGCTGATACTCGCCCGCCTCAGTCCCC
CACCCCCGCAACTCCAGCTGCACAGGATTTCCAACGCAAGCGAAGCTTGATCCG
CCCTGAGAGAAACCGCATTGACCGCGACCATCCAAACTACTATTATCGCCAACA
TGCGGCGAATATGACAGTTTTGCCTTCGACTACGGGAAACGACCCGATTCTAGA
AGATCAAGGAGATATAAATGGCGGGAGTGGCGAGCCGGATCAGCGCAGTCCTC
GGGATGGTCCCTATGGGTCTCCATCGCCTCTGGAATCCCAAGGAACGATGCAAG
GTTCAGACACCGGCAACTTCGAAAAGGACCATAAATCCTCAAAGCTAACTCGTG
ACACAAACAAGTCGAGGAAGCAGACAAGAGAAGAAAGACGCCGCCAGAGGGA
CGCAGAAGTCCTCAAGCCGCCTAGTTTGTGGAACGTGTATTGCTCGATTATCACA
TTCTGGTGCCCTGATTTTATTCTGAGGTGCTTCGGGAAGCCAGCGAAAGAACAAC
AGAGAGCATGGAGGGAGAAGATGGGACTTATCAGCATTATTCTCATGGTCATGG
CCTTCGTCGGCTTTCTCACGTTTGGATTCACTGCCACTGTGTGTGGTAACCCACC
AACCCGACTCCGCGTAAATGAGGTCGACAAAGGATATATGATCTTCCACGGCAA
AGCGTATAATTTATTGGGGGTTCGTCATCCTGCGGCCATGGGTATTCCGGAACAG
TCAAATATCTTGTATGTGCCTGAAAGTCATGGAGGCATGGACGGCAGTTTTCTTT
TCCAAAACGTCAACGGAAAGTGCAAAGGCTTGATCTCAAAGTCAGAGGGATCCG
ACGTTCCAAGTGATGACCAGGGAAACCTCGGTTGGTACTTCCCATGCAACCTTCT
GAACCAGGACGGCTCCAGTGATCCTAAAAAGAAGAGCTTCGGACCCTACCTTGG
CCATCAATGCCATACCACCGCACCTGGACGAACTGCATTCTATGGCATGCACTCT
GCGGGGGATGTCTACTACACCTGGGATGATATCAACAATGGTACAAGGAATTTG
GCTGTCTACTCTGGGAATGTGTTGGACTTGGATCTTCTGAAATGGTTTAATGGGT
CGCAGGTTACTGTCCCTCGAATTTTTGTCGACCTCGGCAACAAGGCTACCACCCT
GAACCAAGGAATCAGAGGTAGAGACATAACACATAAATTCCAAGCCTCTGGAG
ACAAGGAACTTGCCGAATGTTTAGAACAGATCATCAAGGTTGGAAGCGTCGATA
CCGAAACGATTGGCTGTATCGCGTCCCAAGTGGTGCTTTACGTAGCGCTCGCATT
CATCATCTCTATTGTTGGAGCAAAATTCGTCCTCGCTCTCTACTTCCAGTGGTTCT
TGAGCCGCAGGTATGCGGCCAGTAAGACCTCACAATCTTCGGATCCAAAGAAAC
GTAACAGGCAGATCGAGGAGTGGACAAACGACATCTATCGCGCCCCTCCTCGCA
TTGCGGGAGACCCCGGCAGCACTGCTGCTGGTTCCTCGGACAGAAATAGCAGAC
GCGCCAGCAGCATGTTCCTTCCAACCACGTCTCGCTTCACAAGCCCGTATACACT
AAATGGAGAGAAGTCAGGACCACGGCCGGCACCGACGACTATGGCGAGCCAGA
ACTCAGCAGCTCAGCTATTCCCGCCAAACCCGATGTATAGAGGCCAAAACGATA
GCCGAATGAGCTTCCCTAATTCGAACCTAGATGGCGGTGTTATGAGCGATGTATC
AGGCGACGGGCCAGGCCCTGCTGGCTTCATCCATGAAGCTGTCGTGCCTCAGCC
TCCTCCAGAGTGGCAACCATTTGGATATCCTCTTGCACACGCGATCTGCCTTGTC
ACCGCTTACTCAGAAGGTGAGCAGGGTATTAGAACCACACTCGACTCTGTAGCG
ACAACCGATTACCCCAACAGCCACAAGATGATACTCGTTATCTGTGATGGTATG
ATCAAAGGAAAGGGCGAGCTACATTCTACCCCTGAAATTGTCTTGGGCATGATG
AAGGATCACTCTGTGCTGCCTGAGGACGCTCCGGCCTTCAGCTACGTCGCCGTTG
CAAGTGGATCGAAACGACACAACATGGCAAAGGTATATTCTGGATTCTACGATT
ATGGCGCCGACTCGGCAATTCCTTTGGATCGCCAACAGAGAGTGCCCATGATGT
GTCTTGTTAAGTGCGGAACCCCCGATGAAGCATCGAAGAGCAAGCCTGGAAACA
GAGGAAAGCGTGACTCGCAAATTATCCTCATGTCTTTCCTCCAAAAGGTTATGTT
CGACGAGCGGATGACGGAGCTCGAGTTCGAGATGTTCAACGGCTTATGGAAAAT
CACTGGAATGTCACCAGACTTCTTCGAGGTTGTGTTGATGGTTGATGCCGATACC
AAGGTCTTCCCCGACAGCTTAACTCACATGGTTTCCGCCATGGTCAAGGACCCAG
AGATCATGGGATTGTGTGGTGAGACGAAGATTGCAAACAAGAGAGTCTCCTGGG
TTACAGCCATCCAAGTCTTCGAATACTTCATCTCCCATCATCTCTCCAAATCTTTC
GAGTCCGTCTTTGGTGGCGTTACTTGTCTCCCTGGATGTTTCAGTGTGTACCGAAT
CAAGGCACCCAAGGGTGGACAAAACTACTGGGTCCCTATCCTCGCCAACCCTGA
CGTCGTTGAGCACTACTCTGAGAACGTGGTCGATACACTACACAAGAAGAACTT
GTTGTTGCTTGGTGAAGATCGTTACCTCTCAACACTCATGCTCAGGACTTTCCCG
AAGCGCAAGCAGGTCTTTGTGCCACAGGCCGTATGCAAAACTACTGTGCCTGAG
CAATTCTCGGTATTGTTGTCTCAGAGACGTAGATGGATTAACAGTACCATCCACA
ACTTGATGGAACTGGTTCTCGTCAGAGATCTATGTGGTACTTTCTGCTTCAGTAT
GCAGTTCGTGGTGTTCATCGATTTGATCGGAACACTTGTCCTGCCCGCTGCTATT
GCGTTCACTATCTACGTCGTCGTCATATCCATCGTCAGAAAGCCCGTTCAAGTCA
TCCCCCTCGTCCTCCTCGGCCTAATCCTCGGTCTCCCAGCCGTCCTGATCGTCCTC
ACCGCCCACAGATGGTCCTACGTCGTCTGGATGTTAATCTACCTGATCTCCCTCC
CCATCTGGAACTTCGTACTCCCGGTCTACGCCTACTGGAAGTTCGACGACTTCTC
GTGGGGCGACACGCGCAAGACGGCCGGCGAGAAGACCAAGAAGGCCGGCATCG
AGTACGAGGGCGAGTTCGACAGCAGCAAGATCAACATGAAGCGCTGGGGCGAG
TTTGAGAAGGAGAGGAGACAGAGACAGGCCGGGCAGTGGAATGCGCATAGCAA
CATCAGCAGCTATAGGGACGACTACTATGATAATAATAATTAA
CHS3 dsRNA Target (290 bp)
(SEQ ID NO: 14)
GGGCGGCGACCTCCCCCCCCAAGAACGGCGAAATACGTATAGAAGCTCC
ACTGGGCGCCCTCAACCGTCACACGACGCCGAAGTTGGATATGCTGGGCAAGAT
TCGCAATCTCCAACATCTAGACGGACAAGACAACATCGGACAGAACGAGCCGG
GTCTCCCGCTGATACTCGCCCGCCTCAGTCCCCCACCCCCGCAACTCCAGCTGCA
CAGGATTTCCAACGCAAGCGAAGCTTGATCCGCCCTGAGAGAAACCGCATTGAC
CGCGACCATCCAAACTACTATTATC

Sequences Associated with GUS

GUS Gene (1812 bp)
(SEQ ID NO: 15)
ATGTTACGTCCTGTAGAAACCCCAACCCGTGAAATCAAAAAACTCGACGG
CCTGTGGGCATTCAGTCTGGATCGCGAAAACTGTGGAATTGATCAGCGTTGGTG
GGAAAGCGCGTTACAAGAAAGCCGGGCAATTGCTGTGCCGGGCAGTTTTAACGA
TCAGTTCGCCGATGCAGATATTCGTAATTATGTGGGCAACGTCTGGTATCAGCGC
GAAGTCTTTATACCGAAAGGTTGGGCGGGCCAGCGTATTGTACTGCGTTTCGATG
CGGTCACTCATTACGGCAAAGTGTGGGTAAATAATCAGGAAGTGATGGAGCATC
AGGGCGGCTATACGCCATTTGAAGCCGATGTCACTCCGTATGTTATTGCCGGGA
AAAGTGTACGTATCACTGTTTGTGTGAACAACGAACTGAACTGGCAGACTATCC
CGCCGGGAATGGTGATTACCGACGAAAACGGCAAGAAAAAGCAGTCTTACTTTC
ATGATTTCTTTAACTACGCCGGGATCCATCGCAGCGTAATGCTCTACACCACGCC
GAATACCTGGGTGGACGATATCACCGTGGTGACGCATGTCGCGCAAGACTGTAA
CCACGCATCTGTTGACTGGCAGGTGGGGGCAAATGGTGATGTCAGCGTTGAACT
GCGTGATGCGGATCAACAGGTGGTTGCAACTGGACAAGGCACCAGCGGGACTTT
GCAAGTAGTGAATCCGCACCTCTGGCAACCGGGTGAAGGTTATCTCTATGAACT
GTGCGTCACAGCCAAAAGCCAGACAGAGTGTGATATCTACCTGCTGCGCGTCGG
TATCCGGTCAGTGGCAGTGAAGGGCGAACAGTTCCTGATCAACCACAAACCGTT
CTACTTTACTGGCTTTGGTCGTCATGAAGATGCGGATTTGCGCGGCAAAGGATTC
GATAACGTGCTGATGGTGCACGATCACGCACTAATGGACTGGATTGGGGCCAAC
TCCTACCGTACCTCGCATTACCCTTACGCTGAAGAGATGCTCGACTGGGCAGATG
AACATGGCATCGTGGTGATTGATGAAACTGCAGCTGTCGGCTTTAACCTCTCTTT
AGGCATTGGTTTCGAAGCGGGCAACAAGCCGAAAGAACTGTACAGCGAAGAGG
CAGTCAACGGGGAAACCCAGCAGGCGCACTTACAGGCGATTAAAGAGCTGATTG
CGCGTGACAAAAACCACCCAAGCGTGGTGATGTGGAGTATTGCCAACGAACCGG
ATACCCGTCCGCAAGGTGCACGGGAATATTTTGCGCCACTGGCGGAAGCAACGC
GTAAACTCGACCCGACGCGTCCGATCACCTGTGTCAATGTAATGTTCTGCGACGC
TCACACCGATACCATCAGCGATCTCTTTGATGTGCTGTGCCTGAACCGTTATTAC
GGTTGGTATGTCCAAAGCGGCGATTTGGAAACGGCAGAGAAGGTACTGGAAAA
AGAACTTCTGGCCTGGCAGGAGAAACTGCATCAGCCGATTATCATCACCGAATA
CGGCGTGGATACGTTAGCCGGCCTGCACTCAATGTACACCGACATGTGGAGTGA
AGAGTATCAGTGCGCCTGGCTGGATATGTATCACCGCGTCTTTGATCGCGTCAGC
GCTGTCGTCGGTGAGCAGGTATGGAATTTCGCCGATTTTGCGACCTCGCAAGGC
ATATTGCGCGTTGGCGGTAACAAGAAAGGGATCTTCACTCGCGACCGCAAACCG
AAGTCTGCGGCTTTTCTGCTGCAAAAACGCTGGACCGGCATGAACTTCGGTGAA
AAACCGCAGCAGGGAGGCAAACAATGA
GUS dsRNA Construct (237 bp)
(SEQ ID NO: 16)
CAACTCCTACCGTACCTCGCATTACCCTTACGCTGAAGAGATGCTCGACT
GGGCAGATGAACATGGCATCGTGGTGATTGATGAAACTGCAGCTGTCGGCTTTA
ACCTCTCTTTAGGCATTGGTTTCGAAGCGGGCAACAAGCCGAAAGAACTGTACA
GCGAAGAGGCAGTCAACGGGGAAACCCAGCAGGCGCACTTACAGGCGATTAAA
GAGCTGATTGCGCGTGACAAAAACCA

TABLE 4
Exemplary RNA Strands.

| SEQ | Sense/ | Gene | |
|---|---|---|---|
| ID NO | Antisense | Target | Sequence (5′→3′) |
| 17 | Sense | ERG1_1 | CUGCUGCCGUCUCCUUCCGCCGUUCCACCACAU |
| | | | AAAGUACAUAAACACCCUCGUCCAGCUGCGCA |
| | | | GCUUCAACAGCCCGCCCACCACCGCCCAAGUCA |
| | | | GCGUCCCAGCCAAAAUUGCGUCGCCUGUCUCG |
| | | | CGCCCCUUCACCUCCCUCCUCGCUCCCACAAUC |
| | | | GACACCCAGUACCCCUCCAUGACGGCGACGCAG |
| | | | ACCACGCACGAGUCACUCUCGCAGCGGCGCCGA |
| | | | AUGCACCACGAAGCCGAUGUUGUUAUAAU |
| 18 | Antisense | ERG1_1 | AUUAUAACAACAUCGGCUUCGUGGUGCAUUCG |
| | | | GCGCCGCUGCGAGAGUGACUCGUGCGUGGUCU |
| | | | GCGUCGCCGUCAUGGAGGGGUACUGGGUGUCG |
| | | | AUUGUGGGAGCGAGGAGGGAGGUGAAGGGGCG |
| | | | CGAGACAGGCGACGCAAUUUUGGCUGGGACGC |
| | | | UGACUUGGGCGGUGGUGGGCGGGCUGUUGAAG |
| | | | CUGCGCAGCUGGACGAGGGUGUUUAUGUACUU |
| | | | UAUGUGGUGGAACGGCGGAAGGAGACGGCAGC |
| | | | AG |
| 19 | Sense | ERG1_2 | AGCUCACUGUCAUCGCCGACGGGUACAAUUCC |
| | | | AGGUUCCGCAAGCAGAUCAUCAACAAGACUCC |
| | | | CAUUGUUAAGAGCAAGUUUUACGCCUUGACGC |
| | | | UUAUCGACUGCCCCAUGAAGCCUACGAAUUUU |
| | | | GGCCACGUCAUCAUUGGCAAAGCUUUCCCCAU |
| | | | UCUAAUGUACCAGAUUGGCAACCACGAGACGC |
| | | | GCGCGCUA |
| 20 | Antisense | ERG1_2 | UAGCGCGCGCGUCUCGUGGUUGCCAAUCUGGU |
| | | | ACAUUAGAAUGGGGAAAGCUUUGCCAAUGAUG |
| | | | ACGUGGCCAAAAUUCGUAGGCUUCAUGGGGCA |
| | | | GUCGAUAAGCGUCAAGGCGUAAAACUUGCUCU |
| | | | UAACAAUGGGAGUCUUGUUGAUGAUCUGCUUG |
| | | | CGGAACCUGGAAUUGUACCCGUCGGCGAUGAC |
| | | | AGUGAGCU |
| 21 | Sense | ERG_11 | AUCGAGGGACUCUACCAAGAACAGAUCCGCCU |
| | | | CCUAGGCGCCGACCUCCCCCCCUUGACCUAUGA |
| | | | GAACUUGGGCAAGCUCACCCUGAACAACGCCG |
| | | | UCCUCAAGGAGACCCUCCGUCUCCACACCCCCA |
| | | | UCCACUCCAUCCUCCGCAAAGUCAAGUCCCCCA |
| | | | UGCCCGUCCCCGGAACCAACUUCGUCAUCCCAA |
| | | | CAACCCACACCCUCCUCUCCUCC |
| 22 | Antisense | ERG_11 | GGAGGAGAGGAGGGUGUGGGUUGUUGGGAUG |
| | | | ACGAAGUUGGUUCCGGGGACGGGCAUGGGGGA |
| | | | CUUGACUUUGCGGAGGAUGGAGUGGAUGGGGG |
| | | | UGUGGAGACGGAGGGUCUCCUUGAGGACGGCG |
| | | | UUGUUCAGGGUGAGCUUGCCCAAGUUCUCAUA |
| | | | GGUCAAGGGGGGGAGGUCGGCGCCUAGGAGGC |
| | | | GGAUCUGUUCUUGGUAGAGUCCCUCGAU |
| 23 | Sense | ERG24 | GUUGAAGGAACUGAACUGGCUAUUGGAGGACG |
| | | | CUUGAAGUAUAAGUUCAAUACUCUUUCCUCCU |
| | | | CCAUCUUUACCGUCGUUCUCCUCCUCGCCGGUA |
| | | | CUAUCGCGCAGGGCGCCGACUUCCCAGUCUGG |
| | | | ACCUACAUCUGGGACAACUACACCCAGAUCGU |
| | | | CACCGCCAACAUGCUCAUCGCCUUCACCCUCGC |
| | | | AACAUUCGUCUACAUCCGCAGCUUCAGCGUCA |
| | | | AGCCCGGCAACAAAGACAU |
| 24 | Antisense | ERG24 | AUGUCUUUGUUGCCGGGCUUGACGCUGAAGCU |
| | | | GCGGAUGUAGACGAAUGUUGCGAGGGUGAAGG |
| | | | CGAUGAGCAUGUUGGCGGUGACGAUCUGGGUG |
| | | | UAGUUGUCCCAGAUGUAGGUCCAGACUGGGAA |
| | | | GUCGGCGCCCUGCGCGAUAGUACCGGCGAGGA |
| | | | GGAGAACGACGGUAAAGAUGGAGGAGGAAAGA |
| | | | GUAUUGAACUUAUACUUCAAGCGUCCUCCAAU |
| | | | AGCCAGUUCAGUUCCUUCAAC |
| 25 | Sense | CHS2_1 | UCUCAUCUGCACGCAAUUCAUCCUCUCCAUGG |
| | | | GCAACCGGCCACAGGGCGCGAAGAAGUUGUAC |
| | | | CUUUCUAGUAUGGUCAUCUACGCAAUCAUCAU |
| | | | GGUAUACACGACCUUUGCGACGAUCUACAUCG |
| | | | UCGUGCGGCAGUUUACCAGCAACACCAUAACU |
| | | | CUCGGAAACAACAUCUUCACCAACGUCGCCGU |
| | | | GUCCAUCGCCUCGACGCUCGGGCUGUACUUCU |
| | | | UCUCCUCCUUCCUAUACCU |
| 26 | Antisense | CHS2_1 | AGGUAUAGGAAGGAGGAGAAGAAGUACAGCCC |
| | | | GAGCGUCGAGGCGAUGGACACGGCGACGUUGG |
| | | | UGAAGAUGUUGUUUCCGAGAGUUAUGGUGUUG |
| | | | CUGGUAAACUGCCGCACGACGAUGUAGAUCGU |
| | | | CGCAAAGGUCGUGUAUACCAUGAUGAUUGCGU |
| | | | AGAUGACCAUACUAGAAAGGUACAACUUCUUC |
| | | | GCGCCCUGUGGCCGGUUGCCCAUGGAGAGGAU |
| | | | GAAUUGCGUGCAGAUGAGA |
| 27 | Sense | CHS2_2 | UUCGUGUUGGCCCUCGGAAAUAGGCCAAAGGG |
| | | | AUCCCGCUACACUUACAUCACAUCGUUCAUGA |
| | | | UCUUCUCAUUCAUUCAGUUGUAUGUCAUUAUU |
| | | | UUGUCUAUGUUCUUGGUUGUACGCGCGUUUGU |
| | | | CGGACCAAACGUGGACAAAACAGCGAUCUUCA |
| | | | CCACCAAUAUCUUCUCGUCAAACAGCUCCGCU |
| | | | AUCAUUCUCAUCGCGCUGGCGGCCACCUUCGG |
| | | | UUUAUACUUUGUCGCCUCGUUCCUCU |
| 28 | Antisense | CHS2_2 | AGAGGAACGAGGCGACAAAGUAUAAACCGAAG |
| | | | GUGGCCGCCAGCGCGAUGAGAAUGAUAGCGGA |
| | | | GCUGUUUGACGAGAAGAUAUUGGUGGUGAAGA |
| | | | UCGCUGUUUUGUCCACGUUUGGUCCGACAAAC |
| | | | GCGCGUACAACCAAGAACAUAGACAAAAUAAU |
| | | | GACAUACAACUGAAUGAAUGAGAAGAUCAUGA |
| | | | ACGAUGUGAUGUAAGUGUAGCGGGAUCCCUUU |
| | | | GGCCUAUUUCCGAGGGCCAACACGAA |
| 29 | Sense | CHS3 | GGGCGGCGACCUCCCCCCCCAAGAACGGCGAA |
| | | | AUACGUAUAGAAGCUCCACUGGGCGCCCUCAA |
| | | | CCGUCACACGACGCCGAAGUUGGAUAUGCUGG |
| | | | GCAAGAUUCGCAAUCUCCAACAUCUAGACGGA |
| | | | CAAGACAACAUCGGACAGAACGAGCCGGGUCU |
| | | | CCCGCUGAUACUCGCCCGCCUCAGUCCCCCACC |
| | | | CCCGCAACUCCAGCUGCACAGGAUUUCCAACGC |
| | | | AAGCGAAGCUUGAUCCGCCCUGAGAGAAACCG |
| | | | CAUUGACCGCGACCAUCCAAACUACUAUUAUC |
| 30 | Antisense | CHS3 | GAUAAUAGUAGUUUGGAUGGUCGCGGUCAAUG |
| | | | CGGUUUCUCUCAGGGCGGAUCAAGCUUCGCUU |
| | | | GCGUUGGAAAUCCUGUGCAGCUGGAGUUGCGG |
| | | | GGGUGGGGGACUGAGGCGGGCGAGUAUCAGCG |
| | | | GGAGACCCGGCUCGUUCUGUCCGAUGUUGUCU |
| | | | UGUCCGUCUAGAUGUUGGAGAUUGCGAAUCUU |
| | | | GCCCAGCAUAUCCAACUUCGGCGUCGUGUGAC |
| | | | GGUUGAGGGCGCCCAGUGGAGCUUCUAUACGU |
| | | | AUUUCGCCGUUCUUGGGGGGGGAGGUCGCCGC |
| | | | CC |

### References

Berti, C., Fiorini, M., & Sisti, L. (2009). Synthesis of poly(butylene terephtahlate) nanocomposites using anionic clays. European Polymer Journal, 45(1), 70-78.
Bhattacharya, S., Esquivel, B. D., & White, T. C. (2018). Overexpression or Deletion of Ergosterol Biosynthesis Genes Alters Doubling Time, Response to Stress Agents, and Drug Susceptibility in Saccharomyces cerevisiae. mBio, 9(4), e01291-18.
Camacho, C., Coulouris, G., Avagyan, V., Ma, N., Papadopoulos, J., Bealer, K., & Madden, T. L. (2009). BLAST+: Architecture and applications. BMC Bioinformatics, 10(1), 421.
Chaturvedi, S., Rajkumar, S. S., Li, X., Hurteau, G. J., Shtutman, M., & Chaturvedi, V. (2011). Antifungal Testing and High-Throughput Screening of Compound Library against Geomyces destructans, the Etiologic Agent of Geomycosis (WNS) in Bats. PLOS ONE, 6(3), e17032.
Das, G., Deka, B. Kr., Lee, S. H., Park, Y.-B., & Yoon, Y. S. (2015). Poly(vinyl alcohol)/silica nanoparticles based anion-conducting nanocomposite membrane for fuel-cell applications. Macromolecular Research, 23(3), 256-264.
Drees, K. P., Palmer, J. M., Sebra, R., Lorch, J. M., Chen, C., Wu, C.-C., Bok, J. W., Keller, N. P., Blehert, D. S., Cuomo, C. A., Lindner, D. L., & Foster, J. T. (2016). Use of Multiple Sequencing Technologies To Produce a High-Quality Genome of the Fungus Pseudogymnoascus destructans, the Causative Agent of Bat White-Nose Syndrome. Genome Announcements, 4(3), e00445-16.
Dudley, N. R., Amin, A. Z., & Goldstein, B. (2005). Genes required for RNA interference. In K. Appasani (Ed.), RNA Interference Technology (1st ed., pp. 55-68). Cambridge University Press.
Flieger, M., Bandouchova, H., Cerny, J., Chudíčková, M., Kolarik, M., Kovacova, V., Martínková, N., Novák, P., Šebesta, O., Stodůlková, E., & Pikula, J. (2016). Vitamin B2 as a virulence factor in Pseudogymnoascus destructans skin infection. Scientific Reports, 6(1), 33200.
Frick, W. F., Puechmaille, S. J., & Willis, C. K. R. (2016). White-nose syndrome in bats. In Bats in the Anthropocene: Conservation of bats in a changing world (pp. 245-262). Springer.
Gallori, E., Benedetti, E., Bramanti, E., Franchi, M., Orioli, P. L., & Vettori, C. (1996). Studies on the adsorption and binding of nucleic acids on clay minerals. Origins of Life and Evolution of the Biosphere, 26(3-5), 254-255.
Gujjari, A., Rodriguez, B. V., Pescador, J., Maeder, C., Beall, G. W., & Lewis, L. K. (2018). Factors affecting the association of single- and double-stranded RNAs with montmorillonite nanoclays. International Journal of Biological Macromolecules, 109, 551-559.
Hough, J., Howard, J. D., Brown, S., Portwood, D. E., Kilby, P. M., & Dickman, M. J. (2022). Strategies for the production of dsRNA biocontrols as alternatives to chemical pesticides. Frontiers in Bioengineering and Biotechnology, 10, 980592.
Hoyt, J. R., Langwig, K. E., White, J. P., Kaarakka, H. M., Redell, J. A., Parise, K. L., Frick, W. F., Foster, J. T., & Kilpatrick, A. M. (2019). Field trial of a probiotic bacteria to protect bats from white-nose syndrome. Scientific Reports, 9(1), 9158.
Kelley, L. A., Mezulis, S., Yates, C. M., Wass, M. N., & Sternberg, M. J. E. (2015). The Phyre2 web portal for protein modeling, prediction and analysis. Nature Protocols, 10(6), 845-858.
Kumar, R., Kumar, S., Gandhi, M. R., Al-Dhabi, N. A., Paulraj, M. G., & Ignacimuthu, S. (2016). Delivery of chitosan/dsRNA nanoparticles for silencing of wing development vestigial (vg) gene in Aedes aegypti mosquitoes. International Journal of Biological Macromolecules, 86, 89-95.
Lin, Y.-H., Huang, J.-H., Liu, Y., Belles, X., & Lee, H.-J. (2017). Oral delivery of dsRNA lipoplexes to German cockroach protects dsRNA from degradation and induces RNAi response: Oral delivery of dsRNA lipoplexes. Pest Management Science, 73(5), 960-966.
Liu, J., Zhang, Y., Zeng, Q., Zeng, H., Liu, X., Wu, P., Xie, H., He, L., Long, Z., Lu, X., Xiao, M., Zhu, Y., Bo, H., & Cao, K. (2019). Delivery of RIPK4 small interfering RNA for bladder cancer therapy using natural halloysite nanotubes. Science Advances, 5(9), eaaw6499.
Lück, S., Kreszies, T., Strickert, M., Schweizer, P., Kuhlmann, M., & Douchkov, D. (2019). siRNA-Finder (si-Fi) Software for RNAi-Target Design and Off-Target Prediction. Frontiers in Plant Science, 10, 1023.
Malla, S. R. L., Gujjari, A., Corona, C. E., Beall, G. W., & Lewis, L. K. (2023). Spectrophotometric and nucleic acid-binding properties of halloysite clay nanotubes and kaolinite. Heliyon, 9(1), e13009.
Mazu, T., A. Bricker, B., Flores-Rozas, H., & Y. Ablordeppey, S. (2016). The Mechanistic Targets of Antifungal Agents: An Overview. Mini-Reviews in Medicinal Chemistry, 16(7), 555-578.
Nami, S., Baradaran, B., Mansoori, B., Kordbacheh, P., Rezaie, S., Falahati, M., Mohamed Khosroshahi, L., Safara, M., & Zaini, F. (2017). The Utilization of RNA Silencing Technology to Mitigate the Voriconazole Resistance of Aspergillus Flavus; Lipofectamine-Based Delivery. Advanced Pharmaceutical Bulletin, 7(1), 53-59.
Pal, A., Kaur, P., Dwivedi, N., Rookes, J., Bohidar, H. B., Yang, W., Cahill, D. M., & Manna, P. K. (2023). Clay-Nanocomposite Based Smart Delivery Systems: A Promising Tool for Sustainable Farming. ACS Agricultural Science & Technology, 3(1), 3-16.
Palmer, J. M., Drees, K. P., Foster, J. T., & Lindner, D. L. (2018). Extreme sensitivity to μltraviolet light in the fungal pathogen causing white-nose syndrome of bats. Nature Communications, 9(1), 35.
Šečić, E., & Kogel, K.-H. (2021). Requirements for fungal uptake of dsRNA and gene silencing in RNAi-based crop protection strategies. Current Opinion in Biotechnology, 70, 136-142.
Sokolova, V., & Epple, M. (2008). Inorganic Nanoparticles as Carriers of Nucleic Acids into Cells. Angewandte Chemie International Edition, 47(8), 1382-1395.
Yang, Y., Xu, X., Jing, Z., Ye, J., Li, H., Li, X., Shi, L., Chen, M., Wang, T., Xie, B., & Tao, Y. (2022). Genome-Wide Screening and Stability Verification of the Robust Internal Control Genes for RT-qPCR in Filamentous Fungi. Journal of Fungi, 8(9), 952.

While illustrative embodiments have been illustrated and described, it will be appreciated that various changes can be made therein without departing from the spirit and scope of the invention.

The embodiments of the invention in which an exclusive property or privilege is claimed are defined as follows:

1. A double-stranded ribonucleic acid (dsRNA) for inhibiting expression of a target gene in Pseudogymnoascus destructans (Pd), wherein the target gene is selected from the group consisting of a gene involved in the synthesis of ergosterol and a gene involved in the synthesis of chitin, and wherein the dsRNA comprises a first strand comprising a region of complementarity that is substantially complementary to a target region of the mRNA encoded by the target gene.

2. The dsRNA of claim 1, wherein the region of complementarity is from 85% to 100% complementary with the mRNA target region.

3. The dsRNA of claim 1, wherein the target gene is involved in the synthesis of ergosterol.

4. The dsRNA of claim 3, wherein the target gene involved in the synthesis of ergosterol is selected from the group consisting of ERG1_1, ERG1_2, ERG_11, and ERG_24.

5. The dsRNA of claim 4, wherein the mRNA target region is encoded by a target gene sequence comprising or contained within the sequence shown in SEQ ID NO:2, SEQ ID NO:4, SEQ ID NO:6, or SEQ ID NO:8.

6. The dsRNA of claim 5, wherein the RNA sequence of the first strand comprises a sequence having from 95% to 100% identity to the sequence shown in SEQ ID NO:18, SEQ ID NO:20, SEQ ID NO:22, or SEQ ID NO:24.

7. The dsRNA of claim 1, wherein the target gene is involved in the synthesis of chitin.

8. The dsRNA of claim 7, wherein the target gene involved in the synthesis of chitin is selected from the group consisting of CHS2_1, CHS2_2, and CHS3.

9. The dsRNA of claim 8, wherein the mRNA target region is encoded by a target gene sequence contained within the sequence shown in SEQ ID NO:10, SEQ ID NO:12, or SEQ ID NO:14.

10. The dsRNA of claim 9, wherein the RNA sequence of the first strand comprises a sequence having from 95% to 100% identity to the sequence shown in SEQ ID NO:26, SEQ ID NO:28, or SEQ ID NO:30.

11. The dsRNA of claim 1, wherein the dsRNA comprises at least one modified nucleotide.

12. The dsRNA of claim 11, wherein the at least one modified nucleotide is selected from the group consisting of a 2′-O-methyl modified nucleotide and a nucleotide comprising a 5′-phosphorothioate group.

13. The dsRNA of claim 12, wherein the at least one modified nucleotide is selected from the group consisting of a 2′-deoxy-2′-fluoro modified nucleotide, a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, and a non-natural-base-comprising nucleotide.

14. A composition for controlling Pseudogymnoascus destructans (Pd), the composition comprising a dsRNA of claim 1.

15. The composition of claim 14, wherein the dsRNA is bound to a carrier to improve stability and/or delivery of the dsRNA.

16. The composition of claim 15, wherein the carrier is selected from the group consisting of a poly(butylene terephthalate) (PBT) nanocomposite, chitosan, a carbon dot, a silica nanoparticle, montmorillonite, kaolinite, a chitosan nanoparticle, a layered double hydroxide (LDH) nanoparticle, a liposome, a halloysite nanotube, lipofectamine, a nanoclay, an inorganic nanoparticle, a peptide, and a polymer.

17. The composition of claim 14, wherein the composition is formulated for administration by spraying or fogging.

18. The composition of claim 14, wherein the composition comprises two or more dsRNAs for inhibiting expression of two or more Pseudogymnoascus destructans (Pd) target genes.

19. A method for controlling Pseudogymnoascus destructans (Pd) in the environment, the method comprising administering to a substrate comprising Pd, or to a substrate susceptible to establishment of Pd, an effective amount of a composition of claim 14.
